(19) United States
(12) Patent Application Publication
(10) Pub. No.: US 2024/0173413 A1
(43) Pub. Date: May 30, 2024

(54) Ultrasound Responsive Nanobubble Compositions and Methods of Production and Use Thereof

(71) Applicant: University of Central Florida Research Foundation, lnc., Orlando, FL (US)

(72) Inventors: Angela SHAR, Orlando, FL (US); Mehdi RAZAVI, Orlando, FL (US)

(21) Appl. No.: 18/553,307

(22) PCT Filed: Mar. 30, 2022
(86) PCT No.: PCT/US2022/022459

§ 371 Date: Sep. 29, 2023

Related U.S. Application Data
(60) Provisional application No. 63/169,535, filed on Apr. 1, 2021.

(51) Int. Cl. A61K 41/00; A61K 9/51; A61K 38/18; A61K 48/00; A61P 19/10; C12N 15/113

(52) U.S. Cl. CPC A61K 41/0047; A61K 9/5169; A61K 9/5192; A61K 38/1866; A61K 38/1875; A61K 48/0033; A61P 19/10; C12N 15/1137

(57) ABSTRACT

Compositions, methods, and kits relating to ultrasound targeted nanobubble compositions are disclosed. The nanobubble compositions comprise a hollow core containing at least one gas; at least one therapeutic agent disposed in the hollow core; a polymer shell encircling the hollow core; and at least one targeting agent incorporated in the polymer shell. Methods of producing and using the nanobubble compositions are also disclosed.

### CROSS REFERENCE TO RELATED APPLICATIONS/INCORPORATION BY REFERENCE STATEMENT

This application claims benefit under 35 USC § 119(e) of U.S. Provisional Application No. 63/169,535, filed Apr. 1, 2021. The entire contents of the above-referenced patent application(s) are hereby expressly incorporated herein by reference.

### STATEMENT REGARDING FEDERALLY SPONSORED RESEARCH OR DEVELOPMENT Not Applicable.

### BACKGROUND

Osteoporosis is a systemic skeletal disorder characterized by an imbalance between bone-resorbing osteoclasts and bone-forming osteoblasts. A prominent indication is a decrease in bone quality and mass, making people more susceptible to fragility and fracture from low-energy trauma. There are approximately 10 million cases of osteoporosis in the US a year, with an additional 34 million Americans with low bone mass, putting them at a much higher risk for developing osteoporosis. It affects women more than men, where 37.7% of women and 10% of men currently suffer from osteoporosis, not including those with other underlying bone diseases such as spinal osteoarthritis. The US currently spends $10 to $17 billion a year on osteoporosis treatment, and the cost is expected to increase past $22 billion by 2030.

Parathyroid hormone (PTH) therapy is the current alternative to induce anabolic effects on bone formation, which stimulates the mechanism of osteoblasts by binding to a specific receptor and activating signaling pathways. However, various drawbacks have arisen from antiresorptive drugs like bisphosphonate, where its long-term use has been shown to induce jaw osteonecrosis and abnormal long bone fractures. Even as the most clinically used antiresorptive drug, bisphosphonate is not readily absorbed from the gastrointestinal tract, meaning that high doses are necessary, leading to greater gastrointestinal problems. These limitations center around issues of bioavailability and toxicity. Even with PTH therapy, there is possible limited efficacy on nonvertebral bone fractures and activation of bone resorption due to promotion of osteoclastogenesis resulting in chronic exposure that counteracts its anabolic purpose of promoting bone formation. As none of the current therapies for osteoporosis are without adverse effects, innovative therapies such as siRNA therapy for gene silencing are required.

Mammalian cells contain an endogenous RNA interference (RNAi) pathway that is a viable mechanism for regulating signaling pathways within cells by modulating level of gene expression. RNAi can bypass processes in the nucleus and conduct transport through the nuclear envelope. Sequence-specific small interfering RNAs (siRNA) is a portion of the RNAi complex that is able to “silence” specific gene expression having complementary gene strands that are difficult to target with conventional approaches. More particularly, siRNA therapeutic has the ability to target genes based on knowledge of the messenger RNA (mRNA) sequence. The siRNA therapeutic targets and cleaves the complementary mRNA to silence the gene.

However, the clinical translation of siRNA delivery to humans has proven challenging. The pharmacological properties of siRNA include a high anionic charge density (38-50 phosphate groups) and large molecular size (˜13 kDa), both of which make siRNA ineffective in penetrating the cell membrane barrier effectively. Naked siRNA directly injected into the bloodstream or tissue are vulnerable to quick degradation and off-site targeting resulting in immune responses with Toll-like receptors. Furthermore, siRNA administered systemically requires crossing the vascular endothelial barrier before diffusing through the extracellular matrix, while avoiding kidney filtration and non-targeted cell internalization. Even after the siRNA has been up-taken into the targeted cell, they need to be released from endosomal compartments and reunite with the RNAi machinery. Additionally, siRNA must maintain resistance to nuclease degradation to properly function because of their very short half-lives of less than 6 minutes when exposed to serum nucleases. As such, there are significant challenges to using siRNA as a therapeutic and efficient methods of siRNA delivery are needed to realize its full therapeutic potential.

Therapeutic applications of gene-activated matrix, microbubbles (MBs), and nanobubbles (NBs) used as delivery systems are limited due to various disadvantages. For example, using a gene-activated matrix results in the lack of spatiotemporal control. Microbubbles suffer from various disadvantages including low stability due to high solubility of air in blood, requiring large volume for injection, and having a large micrometer size, short circulation time, low injectability, and low cellular uptake. Nanobubbles may generate undesirous reactive oxygen species and have small gas cores which provides less of a contrast agent for imaging.

A number of stimuli-responsive carrier systems currently exist for gene and/or drug delivery. Ultrasound therapy is used for internal tissues and bone healing and is considered safe and non-invasive. Low intensity pulsed ultrasound (LIPUS) and low intensity continuous ultrasound (LICUS) are two methods of ultrasound therapy. LIPUS provides mechanical energy in the form of acoustic pressure waves transmitted through tissue. The rate at which this energy is absorbed is proportional to the density of the tissue it is passing through. LICUS, when used in high intensity, can increase temperature and kill tumor cells. However, the heat produced by the ultrasound beam, in very high intensities, increases the temperature of the target site, which may interfere with the treatment.

Ultrasound therapy has previously been utilized to deliver siRNA into cells through in vitro and in vivo studies into cancer, somatic, and stem cells through the use of delivery systems such as microbubbles and nanobubbles. However, there are many unknown variables and parameters that must be determined in order to utilize ultrasound therapy as a delivery system for other applications.

Other stimuli-responsive nanosystems, such as (but not limited to) pH, reactive oxygen species (ROS), enzyme, redox, thermo, light, and carbon dioxide systems have significant drawbacks due to patient-specific circumstances as well as varying states of disease progression of the target site. For example, pH carriers can result in increased drug/gene release, but there is a large range of pH in gastrointestinal tracts well as patient-specific pH ranges, which makes it difficult to develop a nanoplatform responsive to specific pH values. Redox carriers can increase cytoplasmic release of the drug/gene but they require complex disassembly due to cleavage of thioketal linkages. Enzyme systems have high selectivity but require patient-specific enzymes and disease progression. As such, while these systems may have advantages such as high selectivity and strong correlation with disease states, known endogenous stimuli-responsive drug delivery systems have disadvantages and involve many unknown factors.

Therefore, there is a need in the art for new and improved nanobubble delivery systems, kits containing same, and methods for making and using the nanobubble delivery systems. It is to such improved systems, kits, and methods using a nanobubble platform that the present disclosure is directed.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1. Panel (A) contains a schematic representation of one non-limiting embodiment of a nanobubble synthesis procedure in accordance with the present disclosure. An ultrasonic probe sonicator is utilized to induce PFC phase separation and PFC nanobubble nucleation. The resulting nanobubble formed is then functionalized with alendronate before use. Panels B-H depict the microstructure characterization of nanobubbles (NB) constructed in accordance with the present disclosure. TEM images of NB in low (Panels B-C) and high (Panels D-E) magnification showing the NB size (150±50 nm) and morphology (spherical); (Panels F-G) TEM images of CTSK siRNA-ND-AL showing a liquid core made up of PFC contused with an albumin shell and functionalized with AL. (Panel H) Results of DLS analysis of NB alone, NB-AL, and CTSK siRNA-ND-AL showing the size of 200±50 nm. Panel I depicts the biodegradability of nanobubbles constructed in accordance with the present disclosure. The biodegradation degree (%) in relation to the incubation time (day) up to day 15 can be observed. Statistical analysis: ap<0.05 day 5 vs day 9, 12, and 15; bp<0.05 day 9 vs day 12 and 15; cp<0.05 day 12 vs 15.”

FIG. 2 depicts ultrasound responsiveness of nanobubble compositions constructed in accordance with the present disclosure. The effect of various ultrasound parameters includes waveform (LICUS vs LIPUS), intensity (1 vs 3 W/cm²), and ultrasound exposure time (0-15 min).

FIG. 3 depicts the responsiveness of nanobubble compositions to ultrasound post-exposure time. The induction of LICUS for 5 (Panels A-F) and 9 (Panels G-L) min exposure time resulted in insignificant changes in NB sizes (p>0.05). The length of post-exposure time over 0-180 min showed a significant incremental size increase which led to the complete ultrasound targeted nanobubble expansion and destruction (UTNED) for both 5 (Panels A-F) and 9 (Panels G-L) min exposure times (p<0.05).

FIG. 4 depicts biocompatibility of nanobubble compositions constructed in accordance with the present disclosure. Bright-field microscopy imaging of NB alone (Panels A-B), NB-AL (Panels C-D), CTSK siRNA-ND-AL (Panels E-F), cultured with hBM-MSCs before (Panels A, C, E), and 30 min after induction (Panels B, D, F) at day 0. As a control, hBM-MSCs alone was used (Panel G). The white arrow indicates the presence of NB.

FIG. 5. depicts biocompatibility of nanobubble (NB) compositions constructed in accordance with the present disclosure. Bright-field microscopy imaging of NB alone, NB-AL, CTSK siRNA-ND-AL, cultured with hBM-MSCs at day 7 in low and high magnifications are shown. As a control, hBM-MSCs alone was used.

FIG. 6 depicts nanobubble biocompatibility and internalization. Panels A-L: NB Biocompatibility. Confocal microscopy imaging of cell alone as control (Panels A-C), NB alone (Panels D-F), NB-AL (Panels G-I), CTSK siRNA-ND-AL (Panels J-L), cultured with hBM-MSCs after induction at day 7. Live cells=green (stained with FDA), dead cells=red (stained with PI). Panels M-R: NB Internalization. Confocal microscopy imaging of hBM-MSCs cultured with siRNA CTSK-ND-AL after induction at day 7. Live cells=green (stained with FDA), dead cells=red (stained with PI). The white arrow indicates NB internalized into the cells.

FIG. 7 depicts NB suppression of osteoclastogenesis. Bright-field imaging of hOCP alone (Panels A-D) or hOCP cultured with NB alone (Panels E-F), and CTSK siRNA-ND-AL (Panels G-H) after induction at day 0 at low and high magnifications. The white arrows indicate NB. hOCP with RANKL as a positive control (Panels A-B) and without RANKL as a negative control (Panels C-D) were used.

FIG. 8 depicts use of non-limiting embodiments of nanobubble compositions of the present disclosure in suppression of osteoclastogenesis and treatment of osteoporosis. Panel A contains a schematic representation of ultrasound targeted nanobubble expansion and destruction (UTNED) technology to facilitate CTSK-siRNA delivery into the osteoclasts for treatment of osteoporosis through CTSK gene silencing. The nanobubble is composed of an albumin protein shell with a perfluorocarbon (PFC) gas core containing Cathepsin K (CTSK) siRNA, which was used due to its role in silencing the Cathepsin K gene, which causes osteoporosis. The nanobubble is functionalized on the surface with Alendronate, a molecule that serves as a bone-targeting biomarker to hone osteoclasts. Through the application of ultrasound (probe), the nanobubble grows and expands until it ruptures its contents into the cell, functioning by controlled sequential release where the optimal ultrasound parameters can be modified by optimizing exposure time, intensity, frequency, and waveform to customize to the severity of the disease (e.g., low intensity for superficial fractures vs high intensity for deep lesions). Panels B-J: NB Suppression of Osteoclastogenesis. Bright-field imaging of hOCP cultured with NB alone (Panels B-E), or CTSK siRNA-ND-AL (Panels F-I) after induction at day 7 at low (Panels B-C, F-G) magnifications. The black arrows indicate hOCP and the red arrows hOC. A graph (Panel J) showing a comparison of the osteoclastogenesis suppression between the control and experimental groups.

FIG. 9 demonstrates NB Suppression of Osteoclastogenesis. Confocal microscopy imaging of hOCP alone (Panels A-L) or hOCP cultured with NB alone (Panels M-R), and CTSK siRNA-ND-AL (Panels S-X) after induction at day 7. hOCP with RANKL as a positive control (Panels A-F) and without RANKL as a negative control (Panels G-L) were used. Live cells=green (stained with FDA), dead cells=red (stained with PI). A graph (Panel Y) showing the % cell viability.

FIG. 10 contains a schematic representation of the effect of the nanosystem within bone using ultrasound targeted nanobubble expansion and destruction (UTNED). The femur bone has been magnified to show a comparison between the healthy, osteoporotic, and treated bone. The healthy bone is composed of smaller pores and larger bone density between pores, whereas osteoporosis has significantly reduced bone density and widened the pores. After ultrasound induction at the targeted site, the nanobubble undergoes subsequent expansion and rupture as the CTSK siRNA moves towards the CTSK gene for silencing. Post-treatment, the previously osteoporotic bone takes on the appearance and structure of the treated bone with increased bone growth and reduced porosity.

FIG. 11 graphically depicts the loading and release efficiency of protein from nanobubble compositions constructed in accordance with the present disclosure. Result of Nanodrop spectrophotometry of tryptophan loaded into and released from nanobubbles at 280 nm absorbance.

FIG. 12 depicts Biodistribution of nanobubble compositions constructed in accordance with the present disclosure. In vivo (Panels A-B and D-E) and ex vivo (Panels C and F) X-ray fluorescence imaging of live ovariectomized (OVX) mice at 4 h (Panel D), and 24 h (Panel E) following intravenous injection of CTSK siRNA-NB-AL compared to control mice with no injection (Panels A-B). In vivo images showing accumulation of CTSK siRNA-NB-AL in skeletal system (black arrows indicate jaw, tibia, and femur). (Panel F) Ex vivo imaging of mice organs showed CTSK siRNA-NB-AL accumulation in the femur, tibia, and liver compared to control (Panel C).

FIG. 13 depicts nano-CT results following in vivo administration of nanobubble compositions in accordance with the present disclosure. Representative 3D reconstruction image and nano-CT analysis of bone microarchitecture from the femurs of OVX mice treated with CTSK-siRNA-NB-AL (Panels F-J) vs. controls (Panels A-E). The results show the potential protective effect of CTSK-siRNA-NB-AL in OVX-induced osteoporosis. Red arrows show significant bone loss in the form of pores and cracks.

FIG. 14 depicts histologic results obtained following in vivo administration of nanobubble compositions in accordance with the present disclosure. H&E staining of the femur in the metaphysic area (Panels A-B) and the liver (Panels C-D) for OVX control mice (Panels A, C) and OVX mice treated with CTSK siRNA-NB-AL (Panels B, D).

### DETAILED DESCRIPTION

Before explaining at least one embodiment of the present disclosure in detail, it is to be understood that the present disclosure is not limited in its application to the details of construction and the arrangement of the components or steps or methodologies set forth in the following description or illustrated in the drawings. The present disclosure is capable of other embodiments or of being practiced or carried out in various ways. Also, it is to be understood that the phraseology and terminology employed herein is for the purpose of description and should not be regarded as limiting in any way.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, and/or methods disclosed herein can be made and executed without undue experimentation in light of the present disclosure. While the articles, compositions, kits, and/or methods have been described in terms of particular embodiments, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the terms “about” and “approximately” are used to indicate that a value includes the inherent variation of error for a composition/apparatus/device, the method being employed to determine the value, or the variation that exists among the study subjects. That is, the terms “about” and “approximately” and variations thereof are intended to include not only the exact value qualified by the term, but to also include some slight deviations therefrom, such as deviations caused by measuring error, manufacturing tolerances, wear and tear on components or structures, settling or precipitation of cells or particles out of suspension or solution, chemical or biological degradation of solutions over time, stress exerted on structures, and combinations thereof, for example. In particular, for example, but not by way of limitation, when the term “about” is utilized, the designated value may vary by plus or minus ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a composition, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “patient” includes human and veterinary subjects. In certain embodiments, a patient is a mammal. In certain other embodiments, the patient is a human, including, but not limited to, infants, toddlers, children, young adults, adults, and elderly human populations. “Mammal” for purposes of treatment refers to any animal classified as a mammal, including human, domestic and farm animals, nonhuman primates, and zoo, sports, or pet animals, such as dogs, horses, cats, cows, etc.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, reversing, alleviating, inhibiting the progress of, preventing, or reducing the occurrence of at least one condition, disease, or disorder, or one or more symptoms thereof. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the condition/disease/disorder in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concepts, the present disclosure relates to compositions(s), system(s), and kit(s), as well as methods for making and using same, that are based on an ultrasound targeted nanobubble destruction (UTND) system synthesized using (for example, but not by way of limitation) an in situ sonochemical method. The UTND system can be used as a gene, drug, and/or oxygen delivery system for various conditions, diseases, and disorders, including (but not limited to) various bone conditions, diseases, and disorders, various types of cancer, and various neurodegenerative conditions, diseases, and disorders.

Certain non-limiting embodiments of the present disclosure are directed to an ultrasound responsive targeted nanobubble composition that comprises a hollow core containing at least one gas; a polymer shell encircling the hollow core; at least one therapeutic agent disposed in the hollow core and/or encapsulated within the shell; and at least one targeting agent incorporated in the polymer shell and/or attached to its surface.

Any gas known in the art for use as a contrast agent for ultrasonography may be utilized as the gas present in the hollow core of the nanobubble composition in accordance with the present disclosure. In certain particular (but non-limiting) embodiments, the gas present in the hollow core is a perfluorocarbon (PFC) gas. Non-limiting examples of PFC gases that can be utilized include perfluoropentane (PHP, C₅F₁₂) and perfluorohexane (PFH, C₆F₁₄).

The shell of the nanobubble composition may be formed of any polymer known in the art or otherwise contemplated herein that is useful in forming a polymer shell for ultrasonography reagents. Non-limiting examples of polymers that may be utilized include albumin, poly lactic acid (PLA), poly lactic-co-glycolic acid (PLGA), chitosan, gelatin, and the like, as well as combinations and co-polymers thereof.

In a particular (but non-limiting) embodiment, the polymer shell comprises albumin.

Any therapeutic agents known in the art or otherwise contemplated herein that would benefit from targeted delivery via an ultrasound responsive nanobubble system may be utilized in accordance with the present disclosure. The at least one therapeutic agent may be selected for treatment of various diseases, disorders, or conditions, including (but not limited to) bone conditions, diseases, and disorders (such as, but not limited to, fractures, bone defects, osteoporosis, osteoarthritis, and bone cancer) as well as neurodegenerative diseases (such as, but not limited to, Alzheimer's disease, Parkinson's disease, and amyotrophic lateral sclerosis (ALS)) diseases) and various types of cancer.

In particular (but not by way of limitation), the at least one therapeutic agent may be a gene or other nucleotide-based agent (such as, but not limited to, an siRNA or microRNA), a protein, a drug, and/or a gas (such as, but not limited to, oxygen).

Non-limiting examples of therapeutic agents that may be utilized in accordance with the present disclosure include an siRNA; a microRNA; a gene sequence; an antioxidant; a peptide or protein; CRISPR/CS9 delivery; nanoparticles; extracellular nanovesicles (i.e., exosomes); and drugs; as well as any combinations and/or conjugations thereof. Non-limiting examples of siRNAs that may be utilized in accordance with the present disclosure include Cathepsin K (CTSK) siRNA, VEGF siRNA (for the inhibition of angiogenesis in tumors), histone deacetylase 5 (HDAC5) siRNA, osteoprotegerin (OPG) siRNA, LDL receptor-related protein 5 (LRP5) siRNA, sclerostin siRNA, Noggin siRNA, pyruvate kinase M2 (PKM2) siRNA, doublecortin like kinase 1 (DCLK1) siRNA, and other types of siRNAs for (for example, but not by way of limitation) musculoskeletal disorders and cancer therapies. Non-limiting examples of gene sequences that may be utilized in accordance with the present disclosure include regenerative genes such as vascular endothelial growth factor (VEGF), a bone morphogenetic protein (BMP, such as but not limited to BMP2 or BMP7), hepatocyte growth factor (HGF), and the like. Non-limiting examples of antioxidants that may be utilized in accordance with the present disclosure include vitamins, such as, but not limited to, vitamin C, D, and/or E. Non-limiting examples of proteins and peptides that may be utilized in accordance with the present disclosure include VEGF, a BMP (such as, but not limited to, BMP2 or BMP7), HGF, and the like, as well as any peptide fragments thereof. Non-limiting examples of nanoparticles that may be utilized in accordance with the present disclosure include cerium oxide, mesoporous bioactive glass, bioceramics, ion doped glass-ceramics, and the like. Non-limiting examples of particular combinations of therapeutic agents that may be encapsulated together within the nanobubble compositions of the present disclosure include a combination of two siRNAs, a siRNA/miRNA combination, a siRNA/gene sequence combination, a siRNA/protein or peptide combination, a siRNA/antioxidant combination, a siRNA/CRISPR/CS9 delivery combination, a siRNA/nanoparticle combination, a siRNA/extracellular nanovesicle combination, a siRNA/drug combination, and the like.

In particular (but non-limiting) embodiments, the therapeutic agent is CTSK siRNA. CTSK siRNA can be used to silence genes, such as the Cathepsin K gene which causes osteoporosis. In other particular (but non-limiting) embodiments, the therapeutic agent includes another siRNA for treatment of a bone condition, disease, or disorder.

Alternatively (and/or in addition thereto), the nanobubble compositions of the present disclosure can also be used as oxygen nano shuttles to deliver oxygen to cancer, prevent tumor hypoxia, and improve responsivity to chemo, radiation, or photodynamic therapy. Oxygen delivery can also help with tissue regeneration and wound healing.

Alternatively (and/or in addition thereto), the at least one therapeutic agent may include one or more drugs and/or growth factors for targeted cancer theranostics. Regenerative genes such as (but not limited to) vascular endothelial growth factor (VEGF), bone morphogenetic proteins (BMP), and hepatocyte growth factor (HGF) can be incorporated into the nanobubbles for promoting angiogenesis and tissue regeneration.

In certain particular (but non-limiting) embodiments, the nanobubble composition may include two or more therapeutic agents for codelivery of the two or more therapeutic agents to the same target. When multiple therapeutic agents are present, the agents may be of the same or different classes of molecules. For example (but not by way of limitation), the nanobubble composition may include siRNA and microRNA; siRNA and gene sequence; siRNA and antioxidant; siRNA and protein; etc.

In a particular (but non-limiting) embodiment, the nanobubble composition comprises at least two therapeutic agents that include CTSK siRNA and at least one gene sequence selected from VEGF and a BMP.

The nanobubble composition is functionalized by incorporation within the polymer shell (and/or attachment to the surface of the polymer shell) of one or more targeting agents to hone the nanobubble composition to specific cells/targets at the site or localization of a condition, disorder, or disease to be treated. The targeting agent(s) may be any protein, peptide, or compound capable of honing the nanobubble composition to specific cells/targets at the site or localization of a condition, disorder, or disease to be treated. In some non-limiting embodiments, the at least one targeting agent targets the nanobubble composition to at least a portion of a musculoskeletal system of a subject/patient. For example, but not by way of limitation, the targeting agent can be alendronate, a molecule which serves as a bone-targeting biomarker to hone to osteoclasts. In addition, the nanobubble composition can be functionalized with one or more targeting agent(s) that target to the bone cells or other cell types (for example, but not by way of limitation, at least one of liver, pancreas, neurological, or cancer cells).

In particular (but non-limiting) embodiments, the nanobubble composition may further comprise one or more imaging agents or biomarkers encapsulated inside the nanobubble (i.e., disposed in the hollow core of the nanobubble) and/or incorporated in the polymer shell of the nanobubble in order for the nanobubble to be imageable via various imaging techniques, such as (but not limited to) fluorescent imaging, Raman imaging, and magnetic resonance imaging (MRI). The nanobubbles can also be used for contrast-enhanced ultrasound (CEUS) imaging.

The nanobubble compositions of the present disclosure may be provided with any size that allows the compositions to function as described herein. In particular, the nanobubble compositions should be provided with a size that allows the nanobubble compositions to facilitate cell internalization, easily leak from the vasculature (which can be an advantage for tumor therapy), and/or sequentially release multiple therapeutic agents. NBs are capable of crossing the endothelial gap in blood vessels and enter the interstitial space of cells. The ability of the NB to penetrate into the vasculature and remain after intravenous injection places a high NB concentration due to accumulation in the target site, increasing the permeability of the cell after ultrasound exposure.

For example, but not by way of limitation, the nanobubble composition may be provided with a diameter of about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, and the like, as well as any value that falls between two of the above values (i.e., about 52 nm, about 77 nm, etc.); the nanobubble composition may also have a diameter in a range formed of two of the above values (i.e., a range of from about 20 nm to about 200 nm, a range of from about 20 nm to about 150 nm, a range of from about 15 nm to about 100 nm, a range of from 15 nm to about 50 nm, etc.), as well as a range formed of two values, each of which falls between two of the above values (i.e., a range of from about 22 nm to about 164 nm, a range of from about 47 nm to about 143 nm, etc.).

Certain non-limiting embodiments of the present disclosure are directed to a system that comprises at least one of any of the nanobubble compositions disclosed or otherwise contemplated herein. For example, but not by way of limitation, the system may include two, three, four, five, six, seven, eight, nine, ten, or more different nanobubble compositions. The different nanobubble compositions may contain different therapeutic agents for use in a concurrent therapy treatment plan, and/or the different therapeutic agents may act synergistically with one another. When two or more nanobubble compositions are present in the system, the two or more nanobubble compositions may be designed for administration simultaneously or wholly or partially sequentially.

Certain non-limiting embodiments of the present disclosure are directed to a method of preparing any of the ultrasound responsive targeted nanobubble compositions disclosed or otherwise contemplated herein. The method comprises the steps of: (1) mixing at least one therapeutic agent with at least one gas and at least one polymer to form a first mixture; (2) sonicating the first mixture to form nanobubbles that comprise a polymer shell formed about a hollow core containing the at least one therapeutic agent and the at least one gas; (3) isolating the nanobubbles; and (4) contacting the nanobubbles with a targeting agent to form a second mixture and incubating the second mixture under conditions that allow the targeting agent to be incorporated in the polymer shell of the nanobubbles.

Each of the method steps may be performed at any temperature and for any period of time that allows for the formation of the nanobubble compositions. In certain particular (but non-limiting) embodiments, at least steps (2) and/or (4) can be performed at a temperature in a range of from about 0° C. to about 4° C., and step (4) can be performed for a period in a range of from about 24 hours to about 48 hours.

The nanobubbles can be isolated in step (3) by any methods known in the art. In a particular (but non-limiting) embodiment, step (3) comprises a centrifugation step. Alternatively, the nanobubble size isolation can be performed using a nanofiltration approach.

When the nanobubble composition includes at least one imaging agent, the at least one imaging agent may be added to step (1) and/or to step (4) so that the at least one imaging agent is disposed in the hollow core and/or incorporated in the polymer shell.

Certain non-limiting embodiments of the present disclosure are directed to a method that comprises the step of (1) administering an effective amount of at least one of any of the ultrasound responsive targeted nanobubble compositions disclosed or otherwise contemplated herein to a patient in need thereof.

In certain particular (but non-limiting) embodiments, the method may further comprise the steps of: (2) allowing the nanobubble composition to travel through the patient so that the targeting agent binds to a target within the patient; and (3) exposing the patient to ultrasound, wherein the ultrasound emits acoustic waves based on a determined frequency with an intensity and duration to rupture the nanobubble composition at the target within the patient.

Alternatively, and/or in addition thereto, the method may comprise the step of exposing the patient to ultrasound prior to performing step (1).

In certain particular (but non-limiting) embodiments, the method may include performing steps (1), (2), and (3) above, in combination with step (4): administering an effective amount of a second ultrasound responsive targeted nanobubble composition to a patient in need thereof, wherein the nanobubble composition comprises a hollow core containing at least one gas, a polymer shell encircling the hollow core, at least one therapeutic agent disposed in the hollow core and/or encapsulated within the shell, and at least one targeting agent incorporated in the polymer shell and/or attached to its surface, wherein the at least one targeting agent targets the nanobubble composition to at least a portion of a musculoskeletal system of the patient.

In a particular (but non-limiting) embodiment, the method is further defined as a method of treating a bone condition, disease, or disorder in a patient. For example, but not by way of limitation, the bone condition may be osteoporosis, osteoarthritis, a bone fracture, a bone defect, cancer, and the like.

In another particular (but non-limiting) embodiment, the method is further defined as a method of treating or reducing the occurrence of cancer in a patient.

In another particular (but non-limiting) embodiment, the method is further defined as a method of treating or reducing the occurrence of a neurological disease, disorder, or condition in a patient.

In some non-limiting embodiments, the ultrasound can be selected from low intensity pulsed ultrasound (LIPUS), low intensity continuous ultrasound (LICUS), or focused ultrasound (FUS).

In addition to the use of pulsed or continuous waveforms, the methods of the present disclosure can also utilize any output intensities, frequencies, and exposure times that provide for rupture of the nanobubble compositions and delivery of the at least one therapeutic agent to the target. For example (but not by way of limitation), the method may utilize an output intensity in a range of from about 1 to about 3 W/cm², a frequency in a range of from about 0.5 to about 1.5 MHz, and an exposure time in a range of from about 1 to about 20 minutes. In certain non-limiting embodiments, the LICUS, LIPUS, or FUS can be connected to an imaging probe such as ultrasound, for image-guided nanobubble destruction and therapeutic delivery.

Through application of ultrasound (probe) the nanobubble grows and expands until it ruptures its contents into a target cell. The system functions by controlled sequential release where the ultrasound parameters can be optimized (e.g., exposure time, intensity, frequency, pulsed/continuous waveform, etc.) to customize to the severity of the disease (e.g., low intensity to superficial fractures versus high intensity for deep lesions). Sonoporation and cavitation can be detected within the nanobubble after low-frequency ultrasound is applied. In one non-limiting embodiment, the ultrasound parameters are 3 W/cm² output intensity, 1 MHz frequency, 5 min exposure time, and continuous waveform. However, it will be understood that any ultrasound parameters known in the art or otherwise contemplated herein may be utilized in accordance with the methods of the present disclosure.

Certain non-limiting embodiments of the present disclosure include a kit for preparing an ultrasound responsive targeted nanobubble composition and/or for providing treatment using an ultrasound responsive targeted nanobubble composition. The kit may include one or more of any of the nanobubble compositions disclosed or otherwise contemplated herein. Alternatively, the kit may comprise the various different components of any of the nanobubble compositions disclosed or otherwise contemplated herein. The kit may further include one or more additional components, depending on the consumer.

For example, if the kit is being used in a research lab, the kit may comprise (i) a therapeutic ultrasound probe, and (ii) a nanobubble that does not contain a therapeutic agent or a polymer. An exemplary research lab kit may also comprise (i) a therapeutic ultrasound probe, and (ii) an ultrasound responsive targeted nanobubble composition comprising at least one therapeutic agent disposed in a hollow core of the composition. An exemplary research lab kit may also comprise (i) a therapeutic ultrasound probe, and (ii) an ultrasound responsive targeted nanobubble composition comprising at least one therapeutic agent disposed in a hollow core of the composition, and further comprising a polymer shell encircling the hollow core, and a targeting agent incorporated in the polymer shell. An exemplary research lab kit may also comprise (i) a therapeutic ultrasound probe, and (ii) an ultrasound responsive targeted nanobubble composition comprising a polymer shell encircling the hollow core and a targeting agent incorporated in the polymer shell.

An exemplary research lab kit may also comprise (i) a therapeutic ultrasound probe, and (ii) an ultrasound responsive targeted nanobubble composition comprising CTSK siRNA disposed in a hollow core of the composition. An exemplary research lab kit may also comprise (i) a therapeutic ultrasound probe, and (ii) an ultrasound responsive targeted nanobubble composition comprising CTSK siRNA disposed in a hollow core of the composition, and further comprising a polymer shell encircling the hollow core and alendronate incorporated in the polymer shell. An exemplary research lab kit may also comprise (i) a therapeutic ultrasound probe, and (ii) an ultrasound responsive targeted nanobubble composition comprising a polymer shell encircling the hollow core and alendronate incorporated in the polymer.

If the kit is being used for medical purposes for local delivery, such as in a hospital, where the location already has a therapeutic ultrasound probe, the kit may comprise an ultrasound responsive targeted nanobubble composition comprising at least one therapeutic agent disposed in a hollow core of the composition. An exemplary medical kit may also comprise an ultrasound responsive targeted nanobubble composition comprising at least one therapeutic agent disposed in a hollow core of the composition, and further comprising a polymer shell encircling the hollow core and a targeting agent incorporated in the polymer shell. An exemplary medical kit may also comprise an ultrasound responsive targeted nanobubble composition comprising CTSK siRNA disposed in a hollow core of the composition. An exemplary medical kit may also comprise an ultrasound responsive targeted nanobubble composition comprising CTSK siRNA disposed in a hollow core of the composition, and further comprising a polymer shell encircling the hollow core and alendronate incorporated in the polymer shell.

The kit can further include a set of written or pictorial instructions (or information on how to obtain instructions, either written or pictorial, from the internet) explaining how to use the kit. A kit of this nature can be used in any of the methods described or otherwise contemplated herein.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

### Example 1

In this Example, an ultrasound-responsive targeted nanobubble system for the delivery of osteoporosis-related silencing gene Cathepsin K small interfering RNA (CTSK siRNA) for osteoporosis treatment was developed, optimized, and tested. The nanobubble (NB) is composed of a gas core made from perfluorocarbon, stabilized with albumin, encapsulated with CTSK siRNA, and embedded with alendronate (AL) for bone targeting (CTSK siRNA-NB-AL). Following the development, the responsiveness of CTSK siRNA-NB-AL to a therapeutic ultrasound probe was examined. The results of biocompatibility tests with human bone marrow-derived mesenchymal stem cells proved no significant cell death (p>0.05). When the CTSK siRNA-NB-AL was supplemented with human osteoclast precursors, they suppressed osteoclastogenesis. Thus, this Example establishes the ability of nanotechnology and ultrasound to deliver genes into the osteoclasts. This Example also presents a novel ultrasound responsive and targeted nanobubble platform that can be used as a gene and drug delivery system for various disorders, defects, and diseases, including (but not limited to) cancer.

### Materials and Methods

In Situ Sonochemical Synthesis of Cathepsin K siRNA-loaded Nanobubbles (CTSK siRNA-NB)

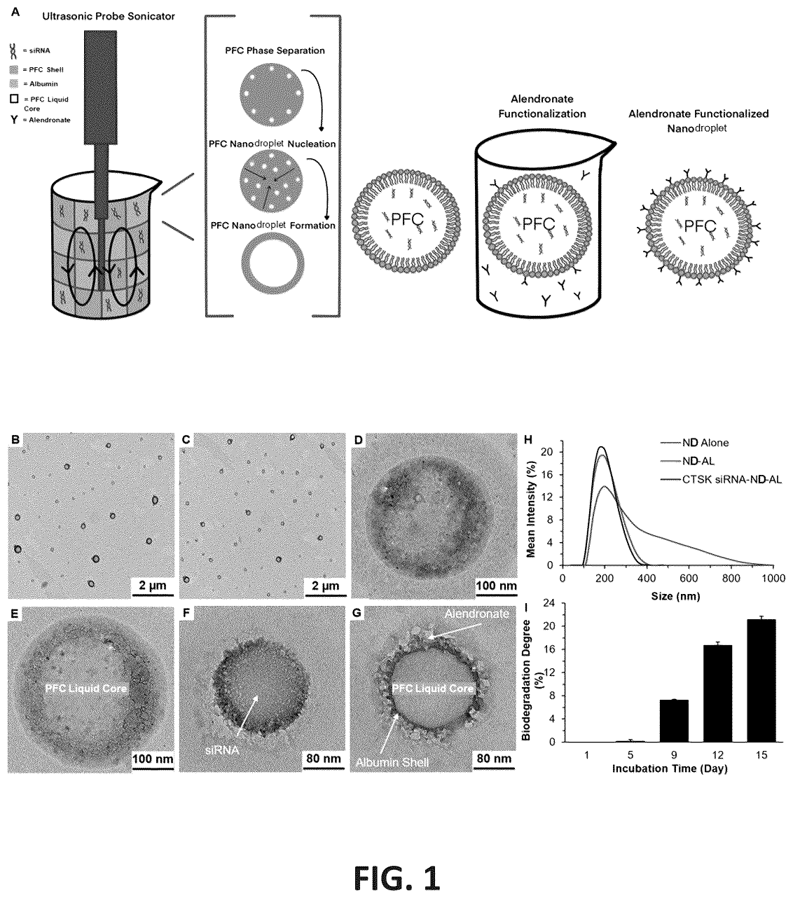

To synthesize CTSK-siRNA-NB, Cathepsin K siRNA (CTSK siRNA; 20 nM, Santa Cruz Biotechnology), mixed with perfluorocarbon (PFC; 300 μL, FluoroMed, such as perfluorohexane (C₆F₁₄)), was added to phosphate buffered saline (PBS; 4 mL, Gibco) containing human serum albumin (HSA; 40 mg, Sigma Aldrich). The mixture was sonicated with an ultrasonic probe (Fisherbrand™ Model 120 Sonic Dismembrator) in an ice bath (30 s/15 s on/off, 200 W, 50% amplitude, 5 times). After a color change of the solution from colorless to white, the obtained emulsion was ultracentrifuged (Optima XPN-100 Ultracentrifuge, Beckman Coulter) at 15,000 rpm for 3 min at 22.5° C. The resulting CTSK siRNA-NB was later washed in PBS three times before dispersion for further analysis. The synthesis procedure can be visualized in Panel A of FIG. 1.

Functionalization of CTSK siRNA-NB with Alendronate (AL) (CTSK siRNA-NB-AL)

Alendronate sodium trihydrate (8.128 mg/mL, 25 mmol, Alfa Aesar) was dispersed in PBS (10 mL). The mixture was placed in a Digital Ultrasonic Cleaner (Digital Pro) for 6 min at 55° C. to increase the solubility of alendronate in PBS. CTSK siRNA-NB (0.015 g) was added to the resulting solution. After 24-48 h at 4° C., CTSK siRNA-NB-AL was obtained and centrifuged at 5,000 rpm for 5 min at 22.5° C. and washed three times with PBS before storing at 4° C. for further use.

### Characterization of Nanobubbles

Nanobubble Imaging with Transmission Electron Microscopy.

Size of the NB alone and CTSK siRNA-NB-AL was measured using Transmission Electron Microscopy (TEM; Jeol JEM-1011) by adding 2.5 μL of NB solution on a copper grid (FCF-3400-CU, Electron Microscopy Sciences-EMS, Hatfield, PA, USA) as described in Razavi, et al. (Nano Lett (2020) 20:7220-9).

### Nanobubble Size Distribution With Dynamic Light Scattering

The average size and size distribution of the NB alone and AL/CTSK siRNA-NB was analyzed using dynamic light scattering (DLS). NB was diluted with PBS (10 mg/mL) to avoid multiscattering and analyzed at 25° C. with a Zetasizer Nano ZS90 (Malvern, UK). The analysis was performed using the following setup: a material refractive index of 1.25; a material absorption of 0.010; a dispersant refractive index of 1.336; and a medium viscosity of 1.05 mPa s. (See Id.

Detection of Surface Charge with Zeta Potential

A Zetasizer Nano ZS90 (Malvern, UK) was used to measure the surface charge (zeta-potential, mV) of NB alone and CTSK siRNA-NB-AL. Measurements represented the average of three batches with 10 runs per measurement. (See Id.)

### Acoustic-Induced Nanobubble Growth and Rupture

Acoustic induction of nanobubbles at 25° C. was performed using an Ultrasonic Therapy Device with 45 mm probe (Win Health Medical Ltd, Unit 1, Oxnam Road Industrial Estate, Jedburgh, TD8 6LS). In order to stimulate nanobubble to grow and rupture, NB (150 mg) were introduced into PBS (1.8 mL) at 25° C. in 6-well plates. The probe was placed underneath the well plate and paired with the plate using Aquasonic Clear® Ultrasound Gel (Parker Laboratories, Inc., 4 Sperry Road, Fairfield, NJ). Sound waves was applied using low-intensity continuous ultrasound therapy (LICUS) and low-intensity pulsed ultrasound therapy (LIPUS). Parameters used for LICUS were the following: output intensity (3 W/cm²), frequency (1 MHz), time (5 min), and waveform (continuous). Parameters for LIPUS were output intensity (3 W/cm²), frequency (1 MHZ), time (5 min), and waveform (pulsed on/off: 1/2). The total time needed to treat the lesion was calculated using the following equation:

Time=(t)×(S)×(PR) (Equation 1)

where t is time (1 min), S is the number of times the ultrasound probe fits onto the lesion, and PR is the pulse ratio [32]. The output intensity parameter was modified for the cell culture studies to prevent premature cell death to 1 W/cm² while other parameters remained constant.

The optimal parameter was determined to be 3 W/cm² output intensity, 1 MHz frequency, 5 min exposure time, and continuous waveform. Following the measurements of nanobubble's surface area as a function of ultrasound post-exposure time, the optimal parameters where the most efficient nanobubble expansion and rupture occurred were obtained after 5 min. The growth and expansion of NB were monitored using optical imaging microscopy (AmScope 40×-2000× Biological Research Microscope with 5.1 MP Camera). Pictures and videos were captured to show progression of the NB's growth and rupture.

### Biodegradability of the Nanobubble

NB alone and AL/CTSK siRNA-NB (100 mg) were suspended in PBS (9.9 mL) at pH 7.4 and incubated at 37° C. The biodegradation degree was determined at day 1, 5, 9, 12, 15 using the following equation:

$$Biodegradation degree = \frac{{W}_{2} - {W}_{1}}{{W}_{1}} \times 100 \qquad ( Equation 2 )$$

where W₁ is the initial weight (mg), and W₂ is the weight at the determined time, i.e., day 1, 5, 9, 12, 15.

Over time, the materials of the nanobubble such as perfluorocarbon, albumin, and alendronate are degraded, causing a change in weight from components of the proteins breaking down into small molecules. The method used to measure biodegradability was using a suspension of nanobubbles in phosphate buffer solution at a pH of 7.4 and incubation at 37° C., followed by a centrifugation to separate the non-degraded from degraded nanobubbles. The dry mass of the nanobubble pellet was weighed at day 1, 5, 9, 12, and 15, and the % biodegradation was reported over incubation time.

Protein Loading and Release from NB

L-tryptophan (Alfa Aesar) was used to measure the loading and release efficiency from the nanobubbles. Tryptophan (204 mg) was mixed with PFC (300 μL), PBS (4 mL), and HSA (40 mg) during the synthesis procedure described above. The tryptophan loaded NB (T-NB) was then centrifuged at 15,000 rpm for 3 min at 22.5° C. to sediment down. The supernatant was gathered carefully to assess the quantity of free tryptophan (unloaded to NB) using a Nanodrop spectrophotometer (Nanodrop® ND-1000). The loading amount of tryptophan could be obtained accordingly. Following the application of LICUS output (3 W/cm² output intensity, 1 MHz frequency, 5 min exposure time, and continuous waveform) on T-NB (15 mg), the T-NB was then centrifuged at 15,000 rpm for 3 min at 22.5° C. The supernatants were removed, and the amount of released tryptophan present in the supernatant was measured using the NanoDrop spectrophotometer. The release efficiency was then determined.

Cytocompatibility of CTSK siRNA-NB-AL

After 1 and 7 days of incubation with CTSK siRNA-NB-AL (0.15 g), the viabilities of human osteoclast precursors (hOCP, Lonza 2T-110, 10,000 cell/well) and human bone marrow derived mesenchymal stem cells (hBM-MSCs, ATCC PCS-500-012, 50,000 cell/well)) were assessed using Live/Dead as described in Id. Cells were labeled using fluorescein diacetate (FDA; for live cells (green fluorescence), Thermo Fisher Scientific, USA) and propidium iodide (PI; for dead cells (red fluorescence), Thermo Fisher Scientific, USA) as the Live/Dead staining solution. The culture medium was removed and the Live/Dead staining solution [FDA (75 μL/well) and PI (75 μL/well)] was added and incubated with cells for 20 min at 37° C./5% C₂. At the end of the incubation time, the staining solution was removed, and the cells were washed three times with PBS. Finally, the live cell imaging solution (Thermo Fisher Scientific, USA) was added to each well (200 μL/well) before imaging. Images were acquired with a Zeiss LSM710 (look at model) Confocal Microscope at a magnification of 10× and figures were created with the FIJI software (ImageJ, GNU General Public License) to quantify live and dead fluorescence in cells and obtain the percentage of live cells.

Osteoclastogenesis of CTSK siRNA-ND-AL

An established protocol for osteoclastogenesis of human osteoclast precursors was used. In brief, human osteoclast precursors (hOCP, Lonza 2T-110, 10,000 cell/well) and osteoclast growth medium (10% FBS, 2mM L-glutamine, 100 units/mL penicillin, and 100 μg/mL streptomycin) were used. Following the culture of hOCP in osteoclast growth media, the cell morphology was observed under a bright field and confocal imaging microscope. As a control for osteoclast differentiation, hOCP with and without receptor activator of nuclear factor κ B ligand (RANKL) was used.

Protein Loading and Release from Nanobubbles

L-tryptophan (Alfa Aesar) was used to measure the loading and release efficiency from my nanobubbles. Tryptophan (204 mg) was mixed with PFC (300 μL), PBS (4 mL), and HSA (40 mg) during the synthesis procedure described above. The tryptophan-loaded ND (T-ND) was then centrifuged at 15,000 rpm for 3 min at 22.5° C. The supernatant was gathered carefully to assess the quantity of free tryptophan (unloaded to ND) using a Nanodrop spectrophotometer (Nanodrop® ND-1000). The loading amount of tryptophan could be obtained accordingly. Following the application of low-intensity continuous ultrasound (LICUS) (3 W/cm² output intensity, 1 MHz frequency, 5 min exposure time, and continuous waveform) on T-ND (15 mg), the T-ND was again centrifuged at 15,000 rpm for 3 min at 22.5° C. The supernatants were removed, and the amount of released tryptophan present in the supernatant was measured using the NanoDrop spectrophotometer. The release efficiency was then determined.

### Statistical Analysis

The following experimental groups were used: NB (n=4 or 5), and CTSK siRNA-NB-AL (n=4), NB-AL (n=4), hBM-MSCs alone (n=4), hOCP alone+RANKL (n=4), and hOCP alone−RANKL (n=4). The results were expressed as mean±standard deviation (SD). Statistical analysis of all quantitative data was performed using a one or two-way ANOVA (analysis of variance) with post hoc Tukey test (Astatsa.com; Online Web Statistical Calculators, USA) or unpaired Student's t test with any differences considered statistically significant when P<0.05 [34].

### Results

### Characterization of Nanobubbles

TEM results showed that the synthesized NB alone (FIG. 1, Panels B-E) and CTSK siRNA-ND-AL (FIG. 1, Panels F-G) were 150±50 nm in size with spherical morphology and a PFC gas core. The existence of CTSK siRNA and AL are also detected in TEM images (FIG. 1, Panels F-G). Using DLS, the NB size was shown to be 200±50 nm (FIG. 1, Panel H). In addition, a single sharp peak in the DLS result shows the uniform spherical shape of NB whereas an irregular shape would have generated two or more peaks as previously shown (34). When testing the zeta potential of NB alone, an average surface charge of −21.05±3 mV was obtained. The AL functionalization significantly changed the NB's zeta potential to −15.9±0.833 mV (p<0.05). However, there was an insignificant change with the loading of CTSK-siRNA in NB-AL at −14.6±0.709 mV (p>0.05).

### Ultrasound Responsiveness of Nanobubbles: Effect of Exposure Time, Intensity, and Waveform

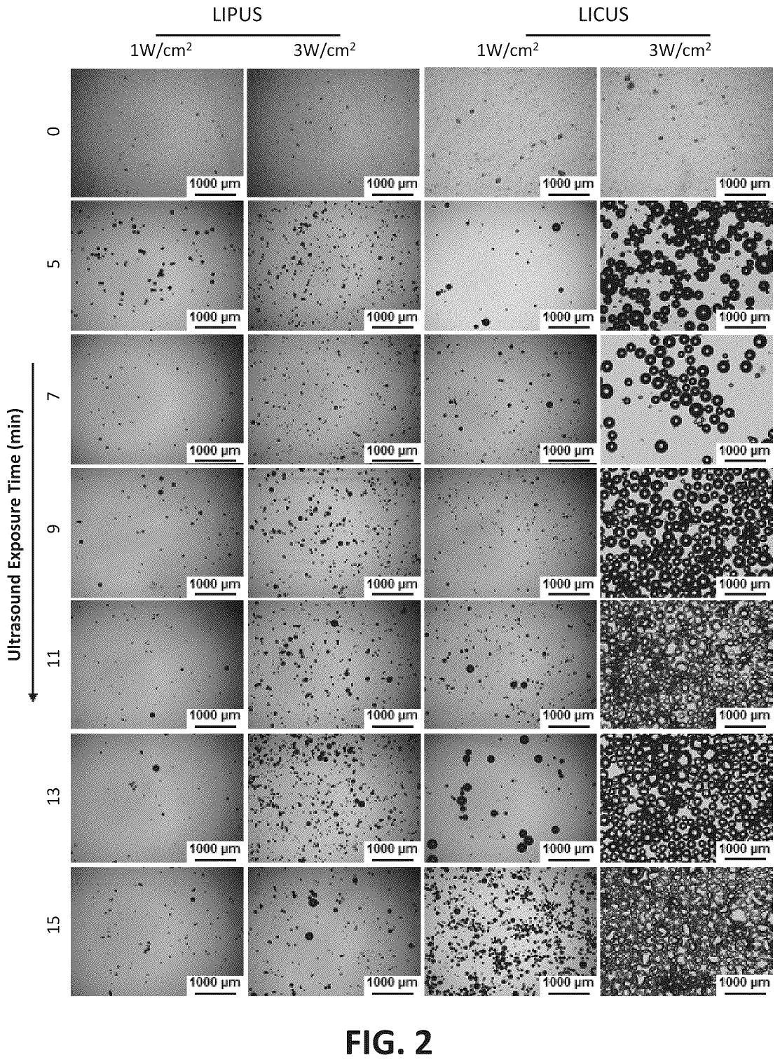

To determine the optimal parameters for ultrasound exposure on nanobubbles, different variables (waveform, intensity, and ultrasound exposure time) were manipulated under controlled conditions. A series of experiments were carried out with varying LICUS and LIPUS waveforms under both 1 W/cm² and 3 W/cm² intensities over 0-15 min time increments of ultrasound exposure time (FIG. 2). The optimal set of parameters should convey the gradual growth, expansion, and rupture of the nanobubbles with the increase in ultrasound exposure time; a criterion most successfully accomplished with LICUS waveform, 3 W/cm², and 1 MHz frequency. From 0-15 min, the nanobubbles under this set of parameters show visible and continuous growth until eventual rupture in a linear progression. This is evident by the increasing size of the nanobubbles from 100 nm at 0 min to 200 nm at 5 min to 500 nm at 13 min until rupture at 15 min, where only the shell remains.

Additionally, the gas core of these nanobubbles expands significantly with increased ultrasound exposure time. At 5 min, the gas core is very small (100 nm), whereas by 13 and 15 min, the core has expanded to 800 and 1000 nm, respectively, to take up the majority of the area inside the nanobubble, leaving a thin outer shell as it is pushed outward. The size increase of NB following ultrasound exposure can be attributed to acoustic droplet vaporization (ADV). This increased tension and increasingly porous shell create a pathway for nanobubble rupture and collapse, providing an efficient method for delivery of genes and drugs via a nanobubble platform.

### Ultrasound Responsiveness of Nanobubbles: Effect of Post-Exposure Time

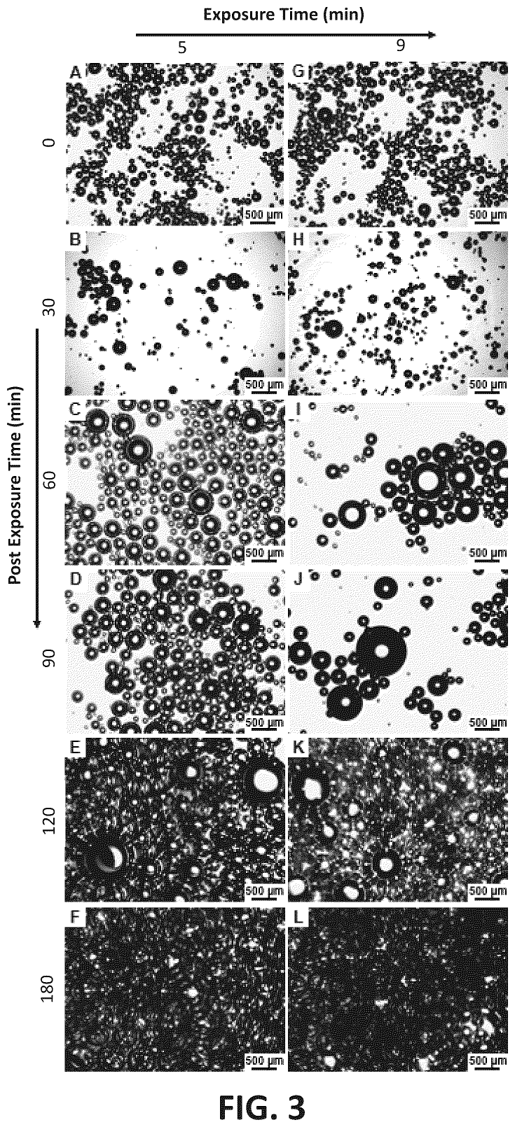

After identifying the optimal ultrasound parameters, the length of exposure time must be further refined to be clinically relevant. To elucidate this information, two time points were utilized: 5 min and 9 min. After these respective lengths of ultrasound exposure time, bright field microscopy images of the nanobubbles were taken over 0-180 min to capture the progressive changes. For both time points, no significant differences were seen during the growth to rupture progression of the nanobubbles (5 min exposure time: 100±10 μm (FIG. 3, Panel A) to 1000±250 μm (FIG. 3, Panel E) to 0±100 μm at collapse (FIG. 3, Panel F) vs 9 min exposure time 120±10 μm (FIG. 3, Panel G) to 1000±250 μm (FIG. 3, Panel J) to 0±50 μm at collapse (FIG. 3, Panel L); p>0.05).

Similar to the previous experiments conducted on the effect of various ultrasound parameters over different ultrasound exposure times on nanobubbles, the porosity of the nanobubble shell intensified as the post ultrasound exposure time increased. This porosity is distinctive when analyzing the size of nanobubbles. As seen in both 5- and 9-min trials (FIG. 3), the nanobubbles grew from 100±10 μm and 120±10 μm, respectively, to 1000±250 μm before collapsing at 0-50 μm. The gas cores in both experiments also reflected the nanobubble's enlargement, as gas cores in Panels E and K of FIG. 3 are noticeably larger at approximately 500 μm (p<0.05).

Based on this data, 5 min of ultrasound exposure time was ultimately selected to prevent prolonged effects. However, in animal and clinical applications, the durability of bone tissue may deem 9 min a more suitable option. Through testing of both 5 and 9 min, the situation presented can be optimized.

### Biodegradability of Nanobubbles

ND alone was suspended in PBS at pH 7.4 and incubated at 37° C. for 15 days. The results showed the biodegradation degree of NB was 0% (day 1), 0.2% (day 5), 7.3% (day 9), 16.8% (day 12), and 21.2% (day 15) (FIG. 1, Panel I).

### Biocompatibility of Nanobubbles

### Cell Morphology

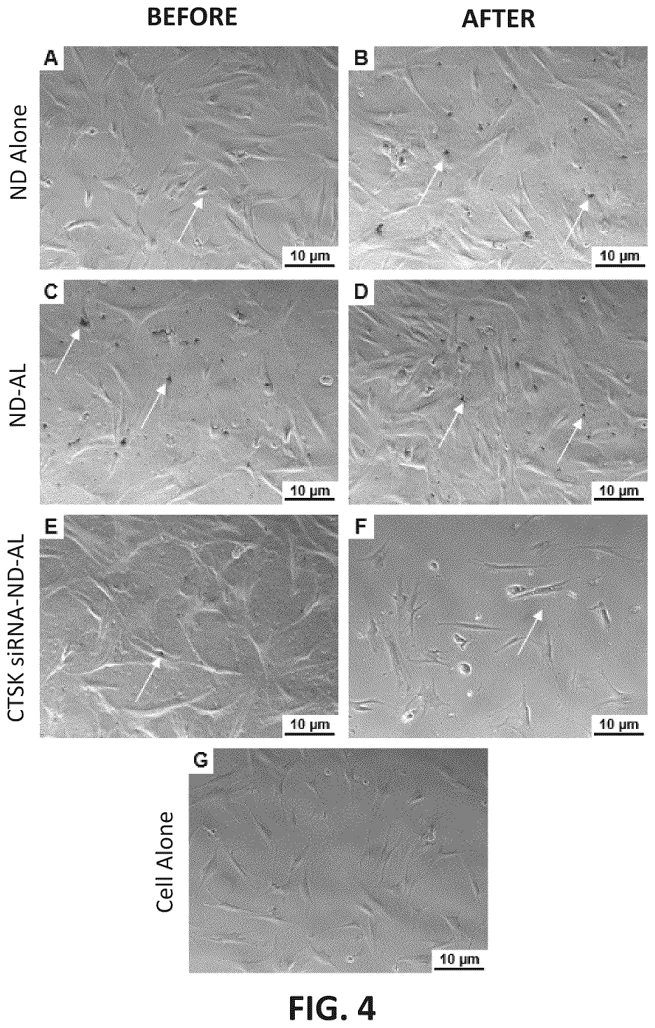

The biocompatibility of the nanobubbles produced herein were tested by culturing with hBM-MSCs. As shown in FIG. 4, the introduction of the nanobubbles had no significant impact (p>0.05) on the morphology of the cells, which retained their spindle-like elongated nature. The nanobubbles, highlighted by the white arrows, are present among the hBM-MSCs (FIG. 4, Panels A-F) and do not appear to cause cell death or decrease cell confluency. The growth and division of the cells remain normal when compared to the cell alone (FIG. 4, Panel G).

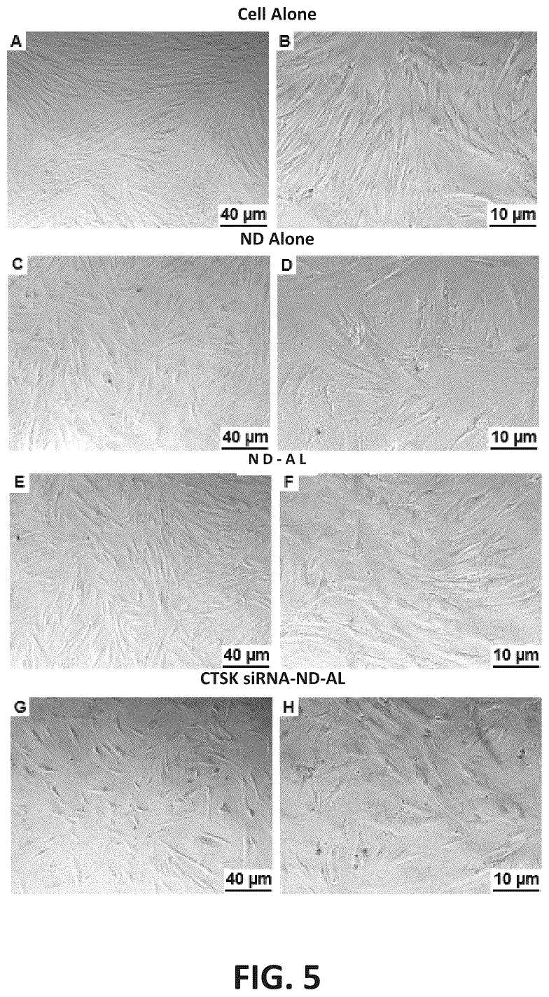

After 7 days of cell culture with hBM-MSCs (FIG. 5), the biocompatibility of the nanobubble trial groups as compared to that of the cell alone trial group continues to be comparable. All experimental groups showed 90-98% confluency and the morphology of the cells continue to be healthy in a spindle-like elongated form.

### Cell Survival

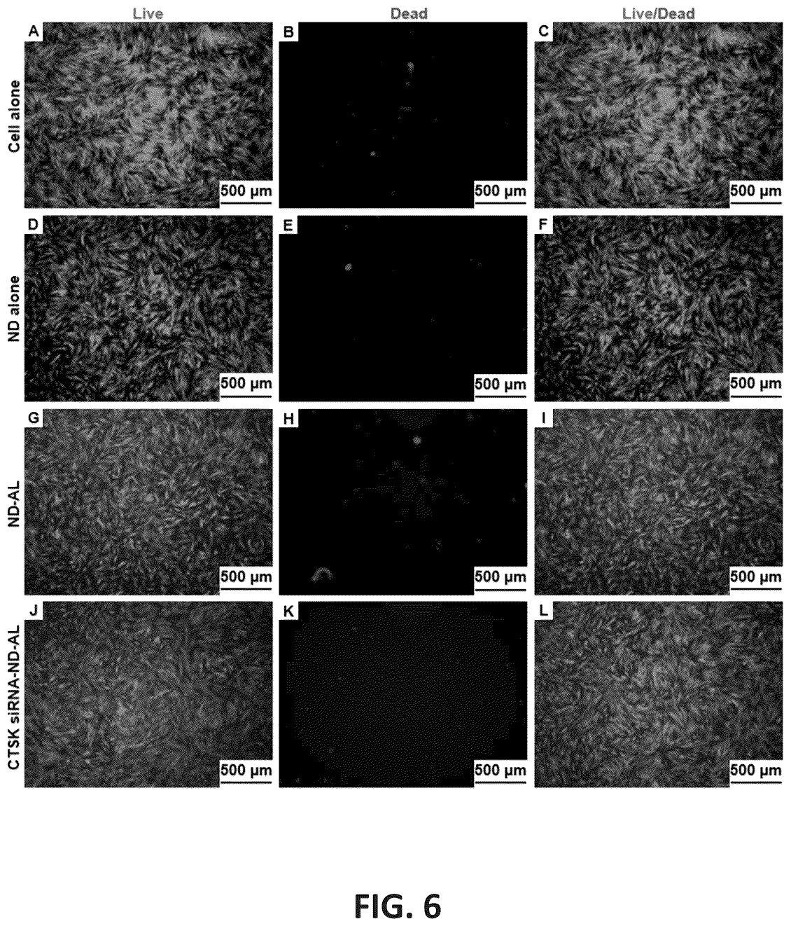

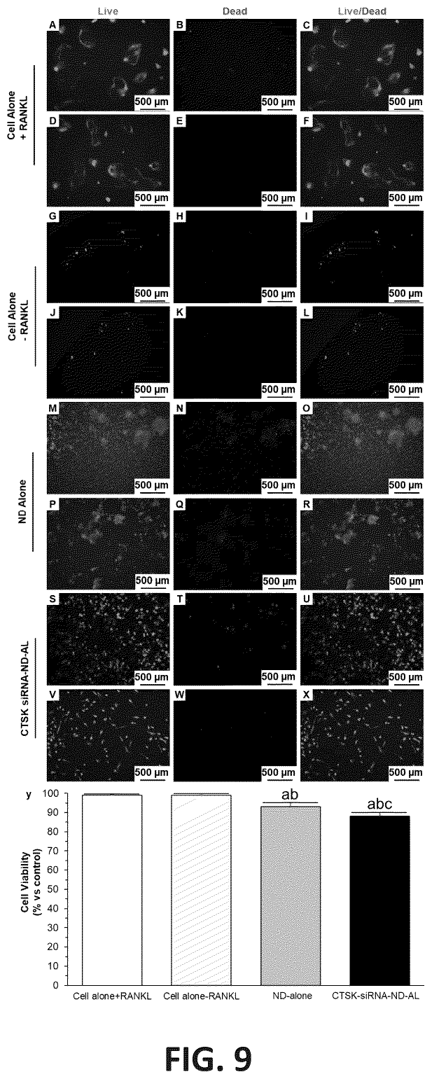

The biocompatibility of the nanobubbles with hBM-MSCs was further verified with confocal microscopy. In FIG. 6, the cell alone group showed 95% confluency (FIG. 6, Panels A-C), NB Alone showed 90% confluency (FIG. 6, Panels D-F), NB-AL showed 98% confluency (FIG. 6, Panels G-I), and CTSK siRNA-ND-AL (FIG. 6, Panels J-L) showed 98% confluency, confirming no drastic changes (p>0.05) to biocompatibility. The number of dead cells (FIG. 6, Panels B, E, H, K) make up less than 1% of the total number of cells, signifying that the nanobubbles pose no threat to the health and growth of hBM-MSCs (FIG. 9, Panel Y).

### Cellular Uptake of Nanobubbles

The internalization of the nanobubbles into hBM-MSCs was visualized in FIG. 6. Although the majority (approximately 95%) of nanobubbles have ruptured after 3 h of ultrasound induction to the nanobubbles, a small portion (approximately 5%) of nanobubbles may not have yet ruptured due to other underlying factors such as the delayed expansion of the nanobubbles because of their very small sizes (<10 nm). These nanobubbles (CTSK siRNA-ND-AL) are shown here internalized into the hBM-MSCs as small spherical dots as indicated by the white arrows (FIG. 6, Panels O, R). With 98-99% confluency, the hBM-MSCs have no notable changes to their morphology.

### Nanobubble Suppression of Osteoclastogenesis

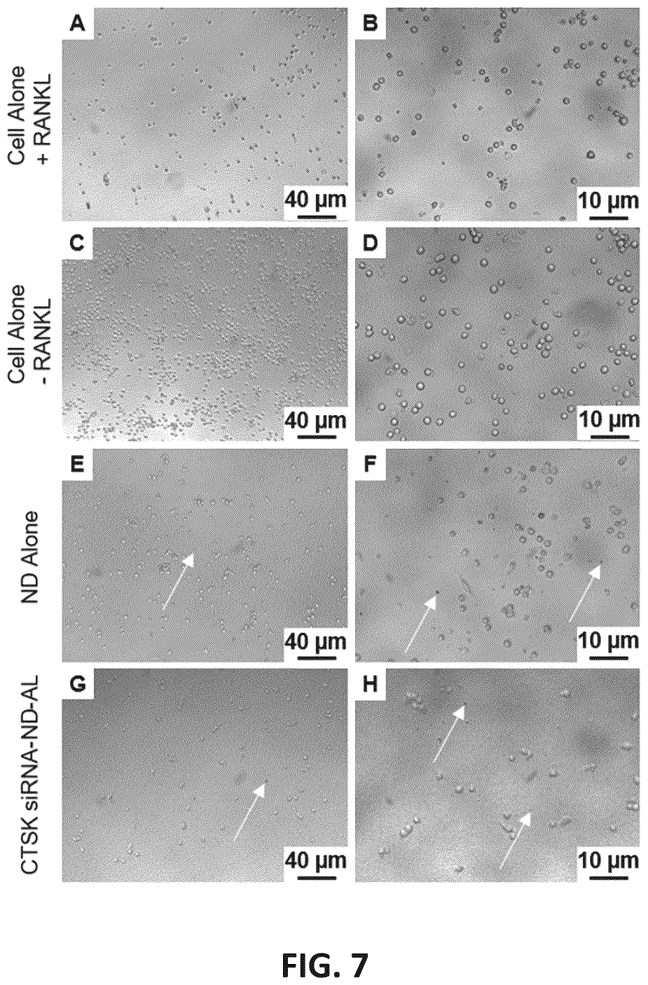

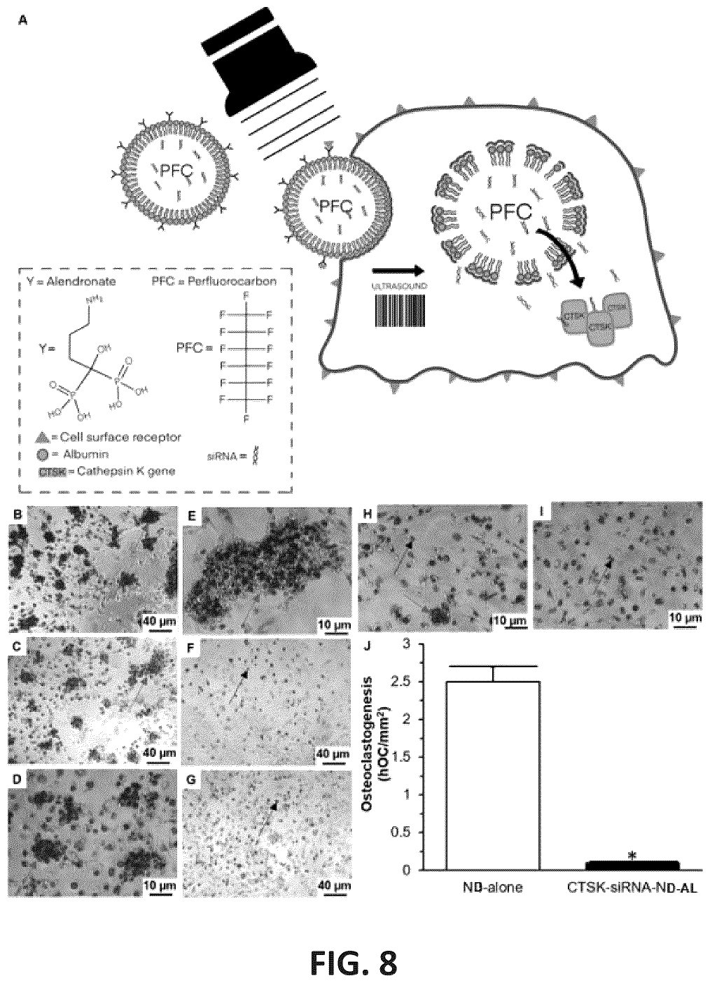

Bright-field microscopy on Day 0 (FIG. 7) shows the presence of nanobubbles (depicted by the white arrows) (FIG. 7, Panels E-H) among the hOCP. The morphology of the hOCP across all experimental groups remains unchanged (spherical). After 7 days, bright field microscopy showed significant differences in cell morphology (p<0.05). For the NB alone (FIG. 8, Panels B-E) group, osteoclasts (shown with red arrows) were seen as large dome-shaped multinucleated cells comprised of fused mononuclear hOCP, similar to that of previous literature for osteoclast morphology (35). This indicates that the presence of NB alone with no Cathepsin K siRNA for downregulation of the Cathepsin K gene does not inhibit the formation of osteoclasts. However, the CTSK siRNA-ND-AL (FIG. 8, Panels F-I) group, had little to no hOC formation and remained in their hOCP state, as shown with black arrows, validating the role of CTSK siRNA in suppressing osteoclastogenesis.

The confocal imaging conducted on Day 7 mirrored the results obtained with bright field imaging with the presence of hOC in the NB alone group (FIG. 9, Panels M-R) and the absence of hOC in the CTSK siRNA-ND-AL group (FIG. 9, Panels S-X). As seen in FIG. 9, there was <1±0.2% cell death in the positive and negative control groups (FIG. 9, Panels A-L), whereas the NB alone group (FIG. 9, Panels M-R) had an average of 7±2% cell death and the CTSK siRNA-ND-AL group (FIG. 9, Panels S-X) had an average 12±2% cell death. NB alone contained an average of 2.5±0.2 hOC/mm²; however, using CTSK siRNA-ND-AL significantly reduced the osteoclastogenesis to 0.1±0.01 hOC/mm² (p<0.05) (FIG. 8, Panel J).

### Protein Loading and Release Efficiency

The Nanodrop spectrophotometer was used to measure the quantity of free tryptophan present in the solution, revealing information about the amount of tryptophan loaded into the nanobubbles (FIG. 11). Since 204 mg of tryptophan was originally used in the synthesis procedure and 89.1 mg of free tryptophan was measured before induction, the resultant loading efficiency was calculated to be 56.3%. After induction (30 min), 184.6 mg of tryptophan was found in the solution, resulting in a 46.8% release efficiency. After induction (1 h), a 100% release efficiency was reached.

Tryptophan was used as a model protein to encapsulate within the nanobubbles due to its ease of detection by a Nanodrop spectrophotometer and cost-effectiveness at higher concentrations. siRNA was difficult to detect unless completely undiluted in which case it is costly. For these reasons, tryptophan was selected to simulate the loading and release efficiency of siRNA.

### Discussion

Existing therapeutic strategies aim to inhibit bone resorption using antiresorptive agents or enhance the formation of bone using anabolic drugs. The antiresorptive drugs suppress osteoclast activity in order to preserve bone mass and increase bone strength; whereas the anabolic drugs attempt to induce bone formation by simultaneously increasing bone mass and reversing bone degradation. Among these antiresorptive agents are bisphosphonates, estrogens, calcium, vitamin D, selective receptor modulators, and denosumab, which are currently clinically available. Bisphosphonate, a common treatment for women 65 and older with a low bone density, costs $66,733 per year which includes a 10-year risk of hip fracture probability that could cost up to $60,000. Furthermore, treating all eligible women with a bisphosphonate would cost $5.6 billion to result in 390,049 fewer fractures. Due to these limitations, novel therapeutics such as RNA therapies must be implemented.

The ability of siRNA therapeutics to target virtually any gene based on knowledge of the messenger RNA (mRNA) sequence provides a strong advantage over traditional drugs. The goal of this therapy is to utilize siRNA to target and cleave the complementary mRNA to silence the gene effectively. The therapeutic potential of using this therapy is promising and can expand to various diseases ranging from osteoporosis to cancers.

The clinical translation of siRNA delivery to humans has proven challenging. The pharmacological properties of siRNA mean that its high anionic charge density (38-50 phosphate groups) and large molecular size (˜13 kDa) make it ineffective in penetrating the cell membrane effectively. Naked siRNA directly injected into the bloodstream or tissue is especially vulnerable to quick degradation and off-site targeting resulting in immune responses with Toll-like receptors. Furthermore, siRNA administered systemically requires crossing the vascular endothelial barrier before diffusing through the extracellular matrix, while avoiding kidney filtration and non-targeted cell internalization. Even after the siRNA has been up-taken into the targeted cell, they need to be released from endosomal compartments and reunite with the RNAi machinery. siRNA must also maintain resistance to nuclease degradation to properly function because of their short half-lives of less than 6 min when exposed to serum nucleases. These measures contribute to the significant challenges that need to be overcome when developing a therapeutic for efficient siRNA delivery.

A primary interest is to regulate the kinetics of siRNA release in a way that the duration of gene silencing can be maintained and/or controlled without the need for repeated treatments. Controlled delivery of nucleic acids ensures that the plasmid DNA is transported to the nucleus for expression and the siRNA is released to eliminate translation of a targeted gene, keeping the siRNA concentration above the critical threshold for a longer length of time. Various osteoporosis-causing genes were carefully considered. Among these, Cathepsin K (CTSK) proved to be the most promising and was therefore studied in this Example. CTSK is a cysteine protease that is predominantly expressed in osteoclasts and has a key role in bone resorption. CTSK readily degrades type I collagen, the major component of the organic bone matrix. With such a major role in the initial process of bone resorption, currently, CTSK is among the most attractive targets for anti-osteoporosis drug development. Although many pharmaceutical companies are working on the development of selective inhibitors for CTSK, there is no FDA approved drug till now. Odanacatib (ODN) is the only CTSK inhibitor candidate which demonstrated high therapeutic efficacy in patients with postmenopausal osteoporosis in Phase III clinical trials, and increased risk of stroke. Unfortunately, the development of ODN was finally terminated due to the cardio-cerebrovascular adverse effects. Therefore, it arouses concerns on the undesirable CTSK inhibition in non-bone sites. It is known that CTSK has far-reaching actions throughout various organs besides bone. Therefore, it would be necessary to design and develop the novel “smart” CTSK inhibitor chemically conjugated with bone-targeted materials, by which it would facilitate the CTSK inhibitor targeting bone to reduce its exposure in non-bone sites so as to prevent the potential adverse effects beyond bone.

The best way to effectively knock-down the CTSK expression in osteoclasts is siRNA. siRNA is a method that introduces short double-stranded RNA molecules that instruct the RNA-induced silencing complex (RISC) to degrade mRNA species complementary to the siRNA. Transfection of siRNA by lipid cations allows for inhibition of expression of the targeted gene. CTSK siRNA has addressed many obstacles affecting other siRNA genes for osteoporosis treatment, such as durability for long term use, lower reactivity but higher potency in biological systems, and specificity to siRNA studies in osteoclasts. The shortcomings of CTSK were also resolved with the use of mouse-specific and human-specific strains of CTSK siRNA. This allowed us to negate any organismal differences in interactions with the siRNA. Using the knowledge gathered, CTSK was therefore chosen as the gene targeted.

While CTSK was the leading gene candidate used in this Example, other genes such as (but not limited to) HDAC5, OPG, LRP5, and/or sclerostin can be targeted with siRNA to reduce bone resorption. In addition, regenerative genes such as vascular endothelial growth factor (VEGF), bone morphogenetic proteins (BMP), and hepatocyte growth factor (HGF) can be incorporated into the nanobubbles for promoting angiogenesis and osteogenesis. Different targeting biomolecules can also be functionalized onto the nanobubbles to improve the skeletal targeting.

The rationale for using ultrasound was due to its effect on nanobubble expansion and destruction for siRNA release, and its ability to cause vibration to the site which induces the formation of vascular networks and vessels while promoting bone healing and regeneration of damaged bone tissue. Perfluorocarbon was used due to its capabilities as a biocompatible and ultrasound-responsive material. It also can function as an oxygen carrier to release oxygen to the tissue, supporting tissue regeneration or oxygenating cancer cells to prevent their hypoxia. Each material and method used were specifically chosen to maximize the positive implications they have on bone tissue.

There are several common delivery systems, such as (but not limited to) microbubbles, gene-activated matrices, and nanodroplets. However, the nanodroplet delivery system was selected to synthesize and test the nanobubble compositions of this Example because i) they can be internalized into the cells due to their smaller sizes compared to microbubbles, ii) they can deliver genes such as the CTSK siRNA used herein on demand (e.g. using ultrasound only), and iii) they are safe, highly stable, and inexpensive with a high shelf-life, iv) they are highly marketable with a low manufacturing price (<$25), low manufacturing time (<1 h), with commercially available chemicals and equipment in a safe and accessible manufacturing environment, and v) there is a wide range of nanobubble sizes providing a great advantage for sequential release of drugs/genes.

The advantages of using nanobubbles (ND) over microbubbles (MB) in this particular study becomes evident in the transport and ease of access to target cells. An intrinsic weakness of MB is their limited application as therapeutics due to their large size. This prevents them from passing into the smaller endothelial gaps of blood vessels, and without this, MB cannot be applied to increase the pervasiveness of the cell membrane to enhance siRNA cellular uptake. However, NB is capable of crossing this endothelial gap and enter the interstitial space of cells, generating greater significance. The ability of the NB to penetrate the vasculature and remain after intravenous injection places a high NB concentration due to accumulation in the target site, increasing the permeability of the cell after ultrasound exposure. Sonoporation and cavitation can be detected within the NB after low-frequency ultrasound is applied.

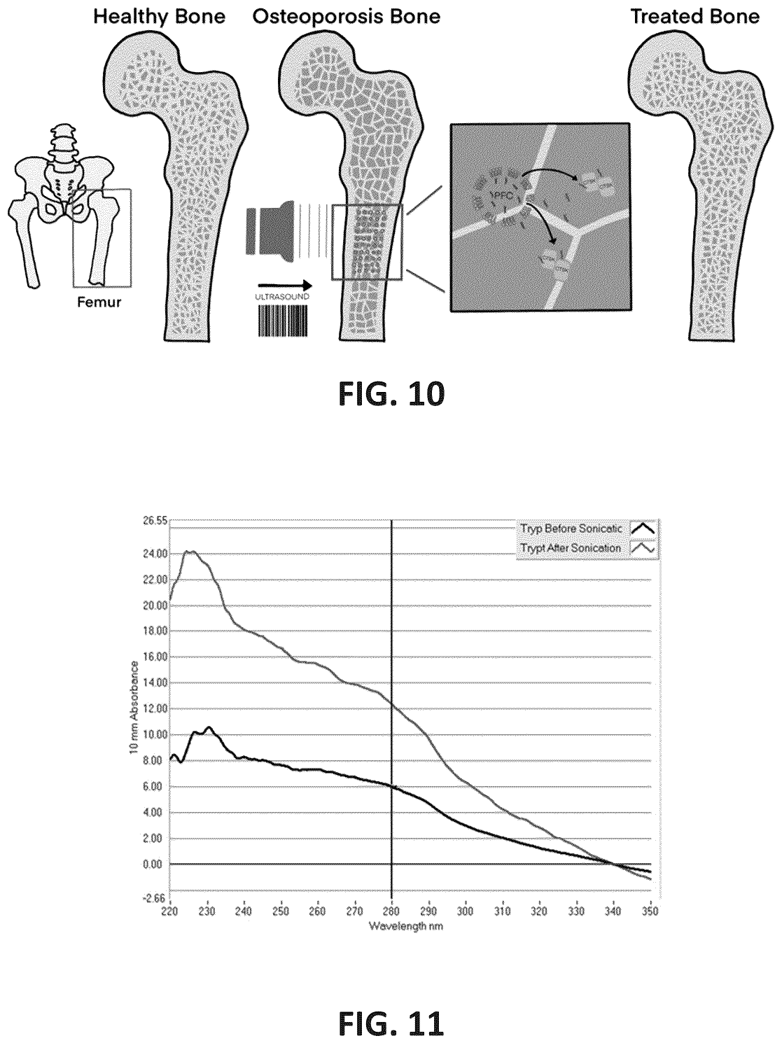

Low-frequency ultrasound exposure delivers high energy to nanobubbles for expansion, inducing holes up to 50 nm in diameter at a half-life of 20-50 ms on the surface of the nanobubble in a process called “sonoporation.” Sonoporation also increases the cellular uptake of drugs and genes that have been administered through ultrasound-targeted nanobubble expansion and destruction (UTNED). Ultrasound-enhanced delivery allows for cavitation of NBs due to the pressure of oscillations, causing the NBs to violently collapse to release siRNA above a specific threshold called inertial cavitation, as seen in FIG. 10. Although it is noted that the heat produced by the beam does increase the temperature, the intensity of the ultrasound used here has an insignificant impact on the temperature (p>0.05).

Certain non-limiting embodiments of the present disclosure are novel because they i) developed and optimized a new sonochemical method for synthesis of targeted ultrasound-responsive nanobubbles, ii) introduced two procedures for therapeutic ultrasound induction including induction of ultrasound at the targeted site or induction before injection of nanobubbles which takes 3 h for 95% rupture of entire nanobubbles, iii) discovered that the disclosed synthesis method can create a broad size range of nanobubbles from 20-150 nm compared to previously synthesized nanobubbles of 400-500 nm. The range of sizes of the nanobubbles produced herein can facilitate cell internalization, easily leak from the vasculature (in case of small nanobubbles <20 nm) which can be an advantage for tumor therapy and sequential release of multiple genes, iv) the previously established nanobubbles have a very small gas core (<20 nm), which does not allow for a high loading capacity into their nanobubble gas cores, v) presents a simple method compared to their complex multi-synthesis procedures, vi) the disclosed nanobubble is the first nanobubble platform that is responsive and compatible with a clinically approved therapeutic ultrasound probe, vii) established a direct correlation between the ultrasound exposure time to nanobubble growth and rupture, as well as ultrasound post-exposure time with nanobubble growth and rupture, viii) discerned the optimal ultrasound parameters by manipulation of exposure time, intensity, frequency, and waveform for tunable expansion and rupture of the nanobubbles for controlled siRNA release, ix) presented an easy manufacturing process (no need for extruder >$3000, polycarbonate membrane >$200, and liquid nitrogen), quick (30 min), in-expensive ($23.30/100 mg), stores easily (4° C.), has a long shelf-time (>3 months), environmentally friendly (no toxic chemicals), functionalizable (targeted molecules; e.g., bisphosphonates as used in this Example), and safe (biocompatible) with cells (e.g., human mesenchymal stem cells and human osteoclasts as used in this Example).

### Conclusion

In summary, an ultrasound responsive targeted nanobubble platform that can deliver CTSK siRNA to osteoclasts has been developed and optimized. The results showed that these nanobubbles can safely reduce osteoclastogenesis and therefore can be used for osteoporosis treatment. This ultrasound responsive targeted nanobubble system has a broader impact in treating other diseases including cancer, neurodegenerative diseases, bone disorders, as well as play a crucial role in oxygen-generating implant technologies and regenerative medicine.

### Example 2

In this Example, the safety and efficacy of the nanobubble compositions of Example 1 to reduce bone resorption and promote bone formation was assessed versus bisphosphonate therapy in OVX mice for osteoporosis treatment.

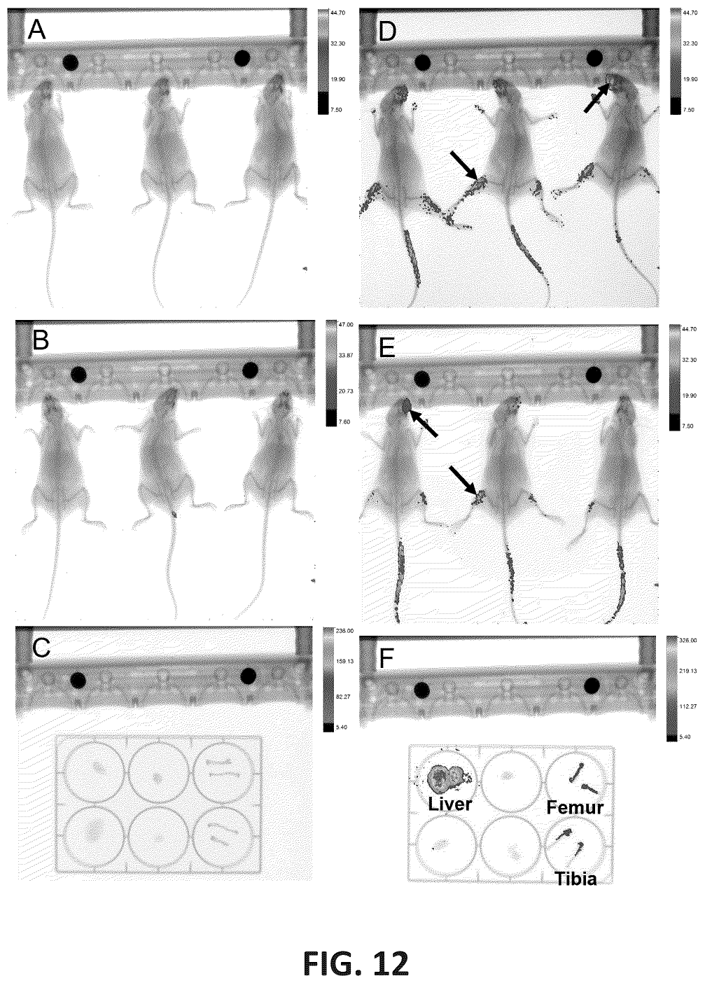

After CTSK siRNA-NB-AL was intravenously injected into OVX mice, no adverse effects, morbidity, or mortality were observed across the study period (6 weeks). As a test of external validity, LIPUS (1 W/cm², 4 min) safety testing showed no adverse effects. X-ray fluorescence imaging of live OVX mice showed accumulation of CTSK siRNA-NB-AL in the skeletal system (FIG. 12, Panels A-B and D-E).

Ex vivo imaging of organs showed CTSK siRNA-NB-AL accumulation in the femur, tibia, liver, and kidney (FIG. 12, Panels C and F).

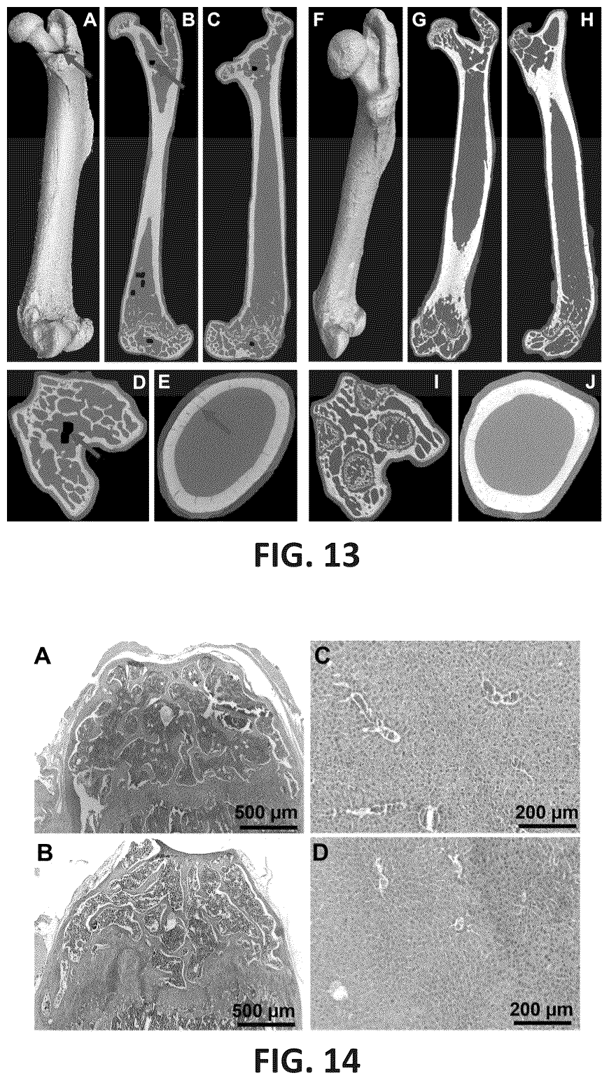

Nano-CT assessments revealed that CTSK siRNA-NB-AL can reduce OVX-related bone loss, as seen by upregulation of trabecular thickness, trabecular number, and cortical thickness in mice, although it requires a quantification. Trabecular separation and bone defects were prevented in the test group versus OVX controls (FIG. 13). Significantly increased bone mass near the femur growth plate was also noted in hematoxylin and eosin (H&E) stained sections of OVX animals treated with CTSK siRNA-NB-AL compared with untreated OVX as controls (FIG. 14, Panels A-B). Despite some residual accumulation in the liver, no sign of histologic damage was detected (FIG. 14, Panels C-D). Taken together, the results demonstrate that CTSK siRNA-NB-AL can protect against bone loss caused by estrogen deficiency.

### NON-ILLUSTRATIVE EMBODIMENTS OF THE INVENTIVE CONCEPTS

Illustrative embodiment 1. An ultrasound responsive targeted nanobubble composition, comprising: a hollow core containing at least one gas; at least one therapeutic agent disposed in the hollow core; a polymer shell encircling the hollow core; and at least one targeting agent incorporated in the polymer shell, wherein the at least one targeting agent targets the nanobubble composition to at least a portion of a musculoskeletal system of a patient.

Illustrative embodiment 2. The nanobubble composition of illustrative embodiment 1, wherein the at least one therapeutic agent comprises an siRNA.

Illustrative embodiment 3. The nanobubble composition of illustrative embodiment 2, wherein the siRNA is selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin siRNA, and combinations thereof.

Illustrative embodiment 4. The nanobubble composition of illustrative embodiment 3, wherein the siRNA is a CTSK siRNA.

Illustrative embodiment 5. The nanobubble composition of any of illustrative embodiments 1-4, wherein the at least one therapeutic agent comprises at least one gene sequence selected from the group consisting of vascular endothelial growth factor (VEGF), bone morphogenetic proteins (BMP), hepatocyte growth factor (HGF), and combinations thereof.

Illustrative embodiment 6. The nanobubble composition of any of illustrative embodiments 1-5, wherein the at least one therapeutic agent comprises CTSK siRNA and at least one gene sequence selected from BMP and VEGF.

Illustrative embodiment 7. The nanobubble composition of any of illustrative embodiments 1-6, wherein the at least one gas is perfluorocarbon gas.

Illustrative embodiment 8. The nanobubble composition of any of illustrative embodiments 1-7, wherein the polymer shell comprises albumin.

Illustrative embodiment 9. The nanobubble composition of any of illustrative embodiments 1-8, wherein the at least one targeting agent comprises alendronate.

Illustrative embodiment 10. The nanobubble composition of any of illustrative embodiments 1, wherein the nanobubble composition has a diameter in a range of from about 20 nm to about 200 nm in diameter.

Illustrative embodiment 11. The nanobubble composition of any of illustrative embodiments 1-10, further comprising at least one imaging agent disposed in the hollow core and/or incorporated in the polymer shell.

Illustrative embodiment 12. A method of preparing an ultrasound responsive targeted nanobubble composition, the method comprising the steps of: (1) mixing at least one therapeutic agent with at least one gas and at least one polymer to form a first mixture; (2) sonicating the first mixture to form nanobubbles that comprise a polymer shell formed about a hollow core containing the at least one therapeutic agent and the at least one gas; (3) isolating the nanobubbles; and (4) contacting the nanobubbles with at least one targeting agent to form a second mixture and incubating the second mixture under conditions that allow the at least one targeting agent to be incorporated in the polymer shell to form the nanobubble composition.

Illustrative embodiment 13. The method of illustrative embodiment 12, wherein the at least one targeting agent targets the nanobubble composition to at least a portion of a musculoskeletal system of a patient.

Illustrative embodiment 14. The method of illustrative embodiment 12 or 13, wherein the at least one targeting agent comprises an siRNA.

Illustrative embodiment 15. The method of illustrative embodiment 14, wherein the siRNA is selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin siRNA, and combinations thereof.

Illustrative embodiment 16. The method of any of illustrative embodiments 12-15, wherein the at least one therapeutic agent comprises at least one gene sequence selected from the group consisting of vascular endothelial growth factor (VEGF), bone morphogenetic proteins (BMP), hepatocyte growth factor (HGF), and combinations thereof.

Illustrative embodiment 17. The method of any of illustrative embodiments 12-16, wherein the at least one therapeutic agent comprises Cathepsin K (CTSK) siRNA, and wherein the at least one targeting agent comprises alendronate.

Illustrative embodiment 18. The method of any of illustrative embodiments 12-17, wherein the at least one therapeutic agent comprises CTSK siRNA and at least one gene sequence selected from BMP and VEGF.

Illustrative embodiment 19. The method of any of illustrative embodiments 12-18, wherein the gas is perfluorocarbon, and the polymer is albumin.

Illustrative embodiment 20. The method of any of illustrative embodiments 12-19, wherein at least one of: step (2) is performed at a temperature in a range of from about 0° C. to about 4° C.; step (3) comprises a centrifugation step; and/or step (4) is performed at a temperature in a range of from about 0° C. to about 4° C. for a period in a range of from about 24 hours to about 48 hours.

Illustrative embodiment 21. The method of any of illustrative embodiments 12-20, wherein the nanobubble composition has a diameter in a range of from about 20 nm to about 200 nm.

Illustrative embodiment 22. The method of any of illustrative embodiments 12-21, wherein at least one imaging agent is added to step (1) and/or (4) so that the at least one imaging agent is disposed in the hollow core and/or incorporated in the polymer shell.

Illustrative embodiment 23. A method, comprising the step of: (1) administering an effective amount of at least one ultrasound responsive targeted nanobubble composition to a patient in need thereof, wherein the nanobubble composition comprises a hollow core containing at least one gas, at least one therapeutic agent disposed in the hollow core, a polymer shell encircling the hollow core, and at least one targeting agent incorporated in the polymer shell, wherein the at least one targeting agent targets the nanobubble composition to at least a portion of a musculoskeletal system of the patient.

Illustrative embodiment 24. The method of illustrative embodiment 23, further comprising the steps of: (2) allowing the nanobubble composition to travel through the patient so that the targeting agent binds to a target within at least a portion of the musculoskeletal system of the patient; and (3) exposing the patient to ultrasound, wherein the ultrasound emits acoustic waves based on a determined frequency with an intensity and duration to rupture the nanobubble composition at the target within the patient.

Illustrative embodiment 25. The method of illustrative embodiment 23 or 24, further comprising the step of exposing the patient to ultrasound prior to performing step (1).

Illustrative embodiment 26. The method of any of Illustrative embodiments 23-25, wherein the at least one targeting agent comprises an siRNA.

Illustrative embodiment 27. The method of illustrative embodiment 26, wherein the siRNA is selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin siRNA, and combinations thereof.

Illustrative embodiment 28. The method of any of illustrative embodiments 23-27, wherein the at least one therapeutic agent comprises at least one gene sequence selected from the group consisting of vascular endothelial growth factor (VEGF), bone morphogenetic proteins (BMP), hepatocyte growth factor (HGF), and combinations thereof.

Illustrative embodiment 29. The method of any of illustrative embodiments 23-28, wherein the at least one therapeutic agent comprises Cathepsin K (CTSK) siRNA, and wherein the targeting agent comprises alendronate.

Illustrative embodiment 30. The method of any of illustrative embodiments 23-29, wherein the gas is perfluorocarbon, and the polymer is albumin.

Illustrative embodiment 31. The method of any of illustrative embodiments 23-30, wherein the nanobubble composition has a diameter in a range of from about 20 nm to about 200 nm.

Illustrative embodiment 32. The method of any of illustrative embodiments 23-31, further comprising the steps of: (2) allowing the nanobubble composition to travel through the patient so that the targeting agent binds to a target within at least a portion of the musculoskeletal system of the patient; (3) exposing the patient to ultrasound, wherein the ultrasound emits acoustic waves based on a determined frequency with an intensity and duration to rupture the nanobubble composition at the target within the patient; and (4) administering an effective amount of a second ultrasound responsive targeted nanobubble composition to a patient in need thereof, wherein the nanobubble composition comprises a hollow core containing at least one gas, at least one therapeutic agent disposed in the hollow core, a polymer shell encircling the hollow core, and at least one targeting agent incorporated in the polymer shell, wherein the at least one targeting agent targets the nanobubble composition to at least a portion of a musculoskeletal system of the patient.

Illustrative embodiment 33. The method of illustrative embodiment 32, wherein in the nanobubble composition of step (1), the at least one therapeutic agent comprises Cathepsin K (CTSK) siRNA, and the targeting agent comprises alendronate, and wherein in the nanobubble composition of step (4), the at least one therapeutic agent comprises at least one gene sequence selected from BMP and VEGF.

Illustrative embodiment 34. The method of any one of illustrative embodiments 23-33, further defined as a method of treating a bone condition, disease, or disorder in the patient.

Illustrative embodiment 35. The method of illustrative embodiment 34, wherein the bone condition, disease, or disorder comprises osteoporosis.

Illustrative embodiment 36. The method of illustrative embodiment 34, wherein the bone condition, disease, or disorder is selected from the group consisting of a bone fracture, a bone defect, osteoarthritis, and a cancer.

Illustrative embodiment 37. The method of any of illustrative embodiments 24-36, wherein the ultrasound is selected from the group consisting of low intensity pulsed ultrasound (LIPUS), low intensity continuous ultrasound (LICUS), and focused ultrasound (FUS).

Illustrative embodiment 38. A kit or system comprising at least one nanobubble composition of any of illustrative embodiments 1-11 and/or at least one nanobubble composition prepared by the method of any of illustrative embodiments 12-22.

Illustrative embodiment 39. The kit or system of illustrative embodiment 38, further defined as comprising at least two nanobubble compositions of any of illustrative embodiments 1-11 and/or at least two nanobubble compositions prepared by the method of any of illustrative embodiments 12-22.

Illustrative embodiment 40. The kit or system of illustrative embodiment 38, further defined as comprising at least three nanobubble compositions of any of illustrative embodiments 1-11 and/or at least three nanobubble compositions prepared by the method of any of illustrative embodiments 12-22.

Illustrative embodiment 41. The kit or system of illustrative embodiment 38, further defined as comprising at least four nanobubble compositions of any of illustrative embodiments 1-11 and/or at least four nanobubble compositions prepared by the method of any of illustrative embodiments 12-22.

Illustrative embodiment 42. The kit or system of illustrative embodiment 38, further defined as comprising at least five nanobubble compositions of any of illustrative embodiments 1-11 and/or at least five nanobubble compositions prepared by the method of any of illustrative embodiments 12-22.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features and steps are recited in the claims and/or disclosed in the specification, these combinations are not intended to limit the disclosure. In fact, many of these features and steps may be combined in ways not specifically recited in the claims and/or disclosed in the specification. Although each dependent claim listed below may directly depend on only one other claim, the disclosure includes each dependent claim in combination with every other claim in the claim set.

What is claimed is:

1. An ultrasound responsive targeted nanobubble composition, comprising:
a hollow core containing at least one gas;
a polymer shell encircling the hollow core;
at least one therapeutic agent disposed in the hollow core and/or encapsulated within the polymer shell; and
at least one targeting agent incorporated in the polymer shell and/or attached to a surface of the polymer shell, wherein the at least one targeting agent targets the nanobubble composition to at least a portion of a musculoskeletal system of a patient.

2. The nanobubble composition of claim 1, wherein the at least one therapeutic agent comprises an siRNA.

3. The nanobubble composition of claim 2, wherein the siRNA is selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin siRNA, and combinations thereof.

4. The nanobubble composition of claim 3, wherein the siRNA is a CTSK siRNA.

5. The nanobubble composition of claim 1, wherein the at least one therapeutic agent comprises at least one gene sequence selected from the group consisting of vascular endothelial growth factor (VEGF), bone morphogenetic proteins (BMP), hepatocyte growth factor (HGF), and combinations thereof.

6. The nanobubble composition of claim 1, wherein the at least one therapeutic agent comprises CTSK siRNA and at least one gene sequence selected from BMP and VEGF.

7. The nanobubble composition of claim 1, wherein the at least one gas is perfluorocarbon gas.

8. The nanobubble composition of claim 1, wherein the polymer shell comprises albumin.

9. The nanobubble composition of claim 1, wherein the at least one targeting agent comprises alendronate.

10. The nanobubble composition of claim 1, wherein the nanobubble composition has a diameter in a range of from about 20 nm to about 200 nm in diameter.

11. The nanobubble composition of claim 1, further comprising at least one imaging agent disposed in the hollow core and/or incorporated in the polymer shell.

12. A method of preparing an ultrasound responsive targeted nanobubble composition, the method comprising the steps of:
(1) mixing at least one therapeutic agent with at least one gas and at least one polymer to form a first mixture;
(2) sonicating the first mixture to form nanobubbles that comprise a polymer shell formed about a hollow core containing the at least one therapeutic agent and the at least one gas;
(3) isolating the nanobubbles; and
(4) contacting the nanobubbles with at least one targeting agent to form a second mixture and incubating the second mixture under conditions that allow the at least one targeting agent to be incorporated in the polymer shell and/or attached to a surface of the polymer shell to form the nanobubble composition.

13. The method of claim 12, wherein the at least one targeting agent targets the nanobubble composition to at least a portion of a musculoskeletal system of a patient.

14. The method of claim 12, wherein the at least one targeting agent comprises an siRNA.

15. The method of claim 14, wherein the siRNA is selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin siRNA, and combinations thereof.

16. The method of claim 12, wherein the at least one therapeutic agent comprises at least one gene sequence selected from the group consisting of vascular endothelial growth factor (VEGF), bone morphogenetic proteins (BMP), hepatocyte growth factor (HGF), and combinations thereof.

17. The method of claim 12, wherein the at least one therapeutic agent comprises Cathepsin K (CTSK) siRNA, and wherein the at least one targeting agent comprises alendronate.

18. The method of claim 12, wherein the at least one therapeutic agent comprises CTSK siRNA and at least one gene sequence selected from BMP and VEGF.

19. The method of claim 12, wherein the gas is perfluorocarbon, and the polymer is albumin.

20. The method of claim 12, wherein at least one of:
step (2) is performed at a temperature in a range of from about 0° C. to about 4° C.;
step (3) comprises a centrifugation step; and/or
step (4) is performed at a temperature in a range of from about 0° C. to about 4° C. for a period in a range of from about 24 hours to about 48 hours.

21. The method of claim 12, wherein the nanobubble composition has a diameter in a range of from about 20 nm to about 200 nm.

22. The method of claim 12, wherein at least one imaging agent is added to step (1) and/or (4) so that the at least one imaging agent is disposed in the hollow core and/or incorporated in the polymer shell.

23. A method, comprising the step of:
(1) administering an effective amount of at least one ultrasound responsive targeted nanobubble composition to a patient in need thereof, wherein the nanobubble composition comprises a hollow core containing at least one gas, a polymer shell encircling the hollow core, at least one therapeutic agent disposed in the hollow core and/or encapsulated within the polymer shell, and at least one targeting agent incorporated in the polymer shell and/or attached to a surface of the polymer shell, wherein the at least one targeting agent targets the nanobubble composition to at least a portion of a musculoskeletal system of the patient.

24. The method of claim 23, further comprising the steps of:
(2) allowing the nanobubble composition to travel through the patient so that the targeting agent binds to a target within at least a portion of the musculoskeletal system of the patient; and
(3) exposing the patient to ultrasound, wherein the ultrasound emits acoustic waves based on a determined frequency with an intensity and duration to rupture the nanobubble composition at the target within the patient.

25. The method of claim 23, further comprising the step of exposing the patient to ultrasound prior to performing step (1).

26. The method of claim 23, wherein the at least one targeting agent comprises an siRNA.

27. The method of claim 26, wherein the siRNA is selected from the group consisting of a Cathepsin K (CTSK) siRNA, histone deacetylase 5 (HDAC5) siRNA, osteoprotegerin (OPG) siRNA, LDL receptor-related protein 5 (LRP5) siRNA, sclerostin siRNA, and combinations thereof.

28. The method of claim 23, wherein the at least one therapeutic agent comprises at least one gene sequence selected from the group consisting of vascular endothelial growth factor (VEGF), bone morphogenetic proteins (BMP), hepatocyte growth factor (HGF), and combinations thereof.

29. The method of claim 23, wherein the at least one therapeutic agent comprises Cathepsin K (CTSK) siRNA, and wherein the targeting agent comprises alendronate.

30. The method of claim 23, wherein the gas is perfluorocarbon, and the polymer is albumin.

31. The method of claim 23, wherein the nanobubble composition has a diameter in a range of from about 20 nm to about 200 nm.

32. The method of claim 23, further comprising the steps of:
(2) allowing the nanobubble composition to travel through the patient so that the targeting agent binds to a target within at least a portion of the musculoskeletal system of the patient;
(3) exposing the patient to ultrasound, wherein the ultrasound emits acoustic waves based on a determined frequency with an intensity and duration to rupture the nanobubble composition at the target within the patient; and
(4) administering an effective amount of a second ultrasound responsive targeted nanobubble composition to a patient in need thereof, wherein the nanobubble composition comprises a hollow core containing at least one gas, at least one therapeutic agent disposed in the hollow core, a polymer shell encircling the hollow core, and at least one targeting agent incorporated in the polymer shell, wherein the at least one targeting agent targets the nanobubble composition to at least a portion of a musculoskeletal system of the patient.

33. The method of claim 32, wherein in the nanobubble composition of step (1), the at least one therapeutic agent comprises Cathepsin K (CTSK) siRNA, and the targeting agent comprises alendronate, and wherein in the nanobubble composition of step (4), the at least one therapeutic agent comprises at least one gene sequence selected from BMP and VEGF.

34. The method of claim 23, further defined as a method of treating a bone condition, disease, or disorder in the patient.

35. The method of claim 34, wherein the bone condition, disease, or disorder comprises osteoporosis.

36. The method of claim 34, wherein the bone condition, disease, or disorder is selected from the group consisting of a bone fracture, a bone defect, osteoarthritis, and a cancer.
